(19) United States
(12) Patent Application Publication
(10) Pub. No.: US 2013/0040852 A1
(43) Pub. Date: Feb. 14, 2013

(54) BIOMARKERS BASED ON A MULTI-CANCER INVASION-ASSOCIATED MECHANISM

(71) Applicant: The Trustees of Columbia University in the City of, New York, NY (US)

(72) Inventors: Dimitris Anastassiou, Tenafly, NJ (US); John Watkinson, Brooklyn, NY (US); Hoon Kim, Houston, TX (US)

(73) Assignee: The Trustees of Columbia University in the City of New York, New York, NY (US)

(21) Appl. No.: 13/650,919

(22) Filed: Oct. 12, 2012

Related U.S. Application Data
(63) Continuation of application No. PCT/US2011/032356, filed on Apr. 13, 2011.
(60) Provisional application No. 61/323,818, filed on Apr. 13, 2010.
(60) Provisional application No. 61/349,684, filed on May 28, 2010.

(51) Int. Cl. C12Q 1/68; G01N 21/64; C40B 30/04

(52) U.S. Cl. 506/9; 435/6.12; 435/6.11

(57) ABSTRACT

The present invention relates to biomarkers which constitute a metastasis associated fibroblast (“MAF”) signature and their use in diagnosing and staging a variety of cancers. It is based, at least in part, on the discovery that identifying the differential expression of certain genes indicates a diagnosis and/or stage of a variety of cancers with a high degree of specificity. In particular, the presence of the signature implies that the cancer has already become invasive. Accordingly, in various embodiments, the present invention provides for methods of diagnosis, diagnostic kits, as well as methods of treatment that include an assessment of biomarker status in a subject. Further, because the differential expression of certain genes can function as marker for the acquisition of metastatic potential, such expression profiles can be used to predict the appropriateness of certain therapeutic interventions, such as the appropriateness of neoadjuvant therapies. Such profiles can also be used to screen for therapeutics capable of inhibiting acquisition of metastatic potential. Accordingly, in various embodiments, the present invention provides for methods of screening therapeutics for their anti-metastatic properties as well as screening kits.

### CROSS REFERENCE TO RELATED APPLICATIONS

This application is a continuation of International Application No. PCT/US2011/032356, filed Apr. 13, 2011 and claims benefit of U.S. Provisional Patent Application No. 61/349,684, filed May 28, 2010 and U.S. Provisional Patent Application 61/323,818, filed Apr. 13, 2010, which are hereby incorporated by reference in their entireties herein.

### I. INTRODUCTION

The present invention relates to the discovery that specific differentially-expressed genes are associated with cancer invasiveness, e.g., invasion of certain cells of primary tumors into adjacent connective tissue during the initial phase of metastasis. The biological mechanism underlying this activity occurs during the course of cancer progression and marks the acquisition of motility and invasiveness associated with metastatic carcinoma. Accordingly, the identification of biomarkers associated with this mechanism, such as the specific differentially-expressed genes disclosed herein, can be used for diagnosing and staging particular cancers, for monitoring cancer progress/regression, for developing therapeutics, and for predicting the appropriateness of certain treatment strategies.

### 2. BACKGROUND OF THE INVENTION

It has been hypothesized that cancer invasiveness is associated with environment of altered proteolysis (Kessenbrock K, Cell 2010; 141:52-67) and can include the appearance of activated fibroblasts. The presence of activated fibroblasts in the “desmoplastic” stroma of tumors, referred to as “carcinoma associated fibroblasts” (CAFs), appear to be part of the biological mechanism underlying cancer invasiveness. As outlined in the present application, the particular subset of CAFs that appear to specifically relate to this metastasis-associated desmoplastic reaction are referred herein as “metastasis associated fibroblasts” (MAFs). Accordingly, herein we refer to the corresponding gene expression signature and biological mechanism that correlates with the presence of such MAFs as “the MAF signature” and “the MAF mechanism,” respectively. There is currently great interest in characterizing the biological mechanism underlying cancer invasion and subsequent metastasis, and this is the problem addressed by the present invention.

### 3. SUMMARY OF THE INVENTION

The present invention relates to biomarkers which constitute a metastasis associated fibroblast (“MAF”) signature and their use in diagnosing and staging a variety of cancers. It is based, at least in part, on the discovery that identifying the differential expression of certain genes indicates a diagnosis and/or stage of a variety of cancers with a high degree of specificity. Accordingly, in various embodiments, the present invention provides for methods of diagnosis, diagnostic kits, as well as methods of treatment that include an assessment of biomarker status in a subject.

The invention is further based, in part, on the discovery that because the differential expression of certain genes can function as marker for the acquisition of invasive potential, such expression profiles can be used to screen for therapeutics capable of inhibiting acquisition of metastatic potential. Accordingly, in various embodiments, the present invention provides for methods of screening therapeutics for their anti-invasion and/or anti-metastatic properties as well as screening kits.

In certain embodiments, the present invention is directed to methods of diagnosing invasive cancer in a subject comprising determining, in a sample from the subject, the expression level, relative to a normal subject, of a COL11A1 gene product wherein overexpression of a COL11A1 gene product indicates that the subject has invasive cancer.

In certain embodiments, the present invention is directed to methods of diagnosing invasive cancer in a subject comprising determining, in a sample from the subject, the expression level, relative to a normal subject, of at least one gene product selected from the group consisting of COL11A1, COL10A1, COL5A1, COL5A2, COL1A1, and COL1A2, and at least one gene product selected from the group consisting of THBS2, INHBA, VCAN, FAP, MMP11, POSTN, ADAM12, LOX, FN1, and SNAI2, wherein overexpression of said gene products indicates that the subject has invasive cancer. In certain of such embodiments, the expression level is determined by a method comprising processing the sample so that cells in the sample are lysed. In certain of such embodiments, the method comprises the further step of at least partially purifying cell gene products and exposing said proteins to a detection agent. In certain of such embodiments, the method comprises the further step of at least partially purifying cell nucleic acid and exposing said nucleic acid to a detection agent. In certain of such embodiments, the method comprises the further step of determining the expression level of SNAI1, where a determination that SNAI1 is not overexpressed and the other gene products are overexpressed indicates that the subject has invasive cancer.

In certain embodiments, the present invention is directed to methods of treating a subject, comprising performing a diagnostic method as outlined above and, where the MAF signature is identified, recommending that the patient undergo an imaging procedure. In certain of such embodiments, the identification of the MAF signature is followed by a recommendation that the patient not undergo neoadjuvant treatment. In certain of such embodiments, the identification of the MAF signature is followed by a recommendation that the patient change their current therapeutic regimen.

In certain embodiments, the present invention is directed to methods for identifying an agent that inhibits cancer invasion in a subject, comprising exposing a test agent to cancer cells expressing a metastasis associated fibroblast signature, wherein if the test agent decreases overexpression of genes in the signature, the test agent may be used as a therapeutic agent in inhibiting invasion of a cancer. In certain embodiments, the metastasis associated fibroblast signature employed in method comprises overexpression of at least one gene product selected from the group consisting of COL11A1, COL10A1, COL5A1, COL5A2, COL1A1, and COL1A2, and at least one gene product selected from the group consisting of THBS2, INHBA, VCAN, FAP, MMP11, POSTN, ADAM12, LOX, FN1, and SNAI2.

In certain embodiments, the present invention is directed to kits comprising: (a) a labeled reporter molecule capable of specifically interacting with a metastasis associated fibroblast signature gene product; (b) a control or calibrator reagent, and (c) instructions describing the manner of utilizing the kit.

In certain embodiments, the present invention is directed to kits comprising: (a) a conjugate comprising an antibody that specifically interacts with a metastasis associated fibroblast signature antigen attached to a signal-generating compound capable of generating a detectable signal; (b) a control or calibrator reagent, and (c) instructions describing the manner of utilizing the kit. In certain of such embodiments, the present invention is directed to kits comprising: a metastasis associated fibroblast signature antigen-specific antibody, where the metastasis associated fibroblast signature antigen bound by said antibody comprises or is otherwise derived from a protein encoded by one or more of the following genes: COL11A1, COL10A1, COL5A1, COL5A2, COL1A1, COL1A2, THBS2, INHBA, VCAN, FAP, MMP11, POSTN, ADAM12, LOX, FN1, and SNAI2

In certain embodiments, the present invention is directed to kits comprising: (a) a nucleic acid capable of hybridizing to a metastasis associated fibroblast signature nucleic acid; (b) a control or calibrator reagent; and (c) instructions describing the manner of utilizing the kit. In certain of such embodiments, the kids comprise: (a) a nucleic acid sequence comprising: (i) a target-specific sequence that hybridizes specifically to a metastasis associated fibroblast signature nucleic acid, and (ii) a detectable label; (b) a primer nucleic acid sequence; (c) a nucleic acid indicator of amplification; and. (d) instructions describing the manner of utilizing the kit. In certain of such embodiments, the present invention is directed to kits comprises a nucleic acid that hybridizes specifically to a metastasis associated fibroblast signature nucleic acid comprising or otherwise derived from one of the following genes: COL11A1, COL10A1, COL5A1, COL5A2, COL1A1, COL1A2, THBS2, INHBA, VCAN, FAP, MMP11, POSTN, ADAM12, LOX, FN1, and SNAI2.

### 4. DESCRIPTION OF THE FIGURES

FIG. 1: Illustration of the general steps of particular, non-limiting, embodiments of the present invention.

FIG. 2: Evaluation of the EVA metric for gene COL11A1 in the TCGA ovarian cancer data set using phenotypic staging threshold the transition to stage IIIc

FIG. 3: Illustration for the low-complexity implementation of the EVA algorithm.

FIG. 4. The pseudo-code for the mechanistic unbiased (only dependent on the phenotype) algorithm described in the Example.

### 5. DETAILED DESCRIPTION OF THE INVENTION

5.1. Identification of the MAF Signature

A study (Bignotti E, Am J Obstet Gynecol 2007; 196:245 e1-11) of serous papillary ovarian carcinomas, comparing the gene expression profiles of 14 samples of primary and 17 samples of omental metastatic tumors, identified 156 differentially expressed genes. To investigate the significance of these genes in an independent rich dataset we performed hierarchical clustering, using only these 156 genes, on The Cancer Genome Atlas (TCGA) gene expression dataset consisting of 377 ovarian cancer samples containing precise staging information. The resulting heat map revealed a prominent “red square” of about 100 highly overexpressed genes in 94 samples Remarkably, none of the 41 samples from tumors of stages IIIb and below were among the 94 “red square” samples (P=4×10-6), consistent with coordinated overexpression of these genes indicating that a tumor has progressed into at least stage IIIc.

To determine whether this behavior would be exhibited by genes in other cancers, we developed a computational technique, which identifies, in an unbiased manner, coordinately overexpressed genes associated with a particular phenotype (such as transition to a particular stage). Our results consistently “rediscover” the same “core” signature of overexpressed genes. We found that this phenomenon occurs in multiple cancers, each of which has its own features potentially involving additional genes, but the core signature is common.

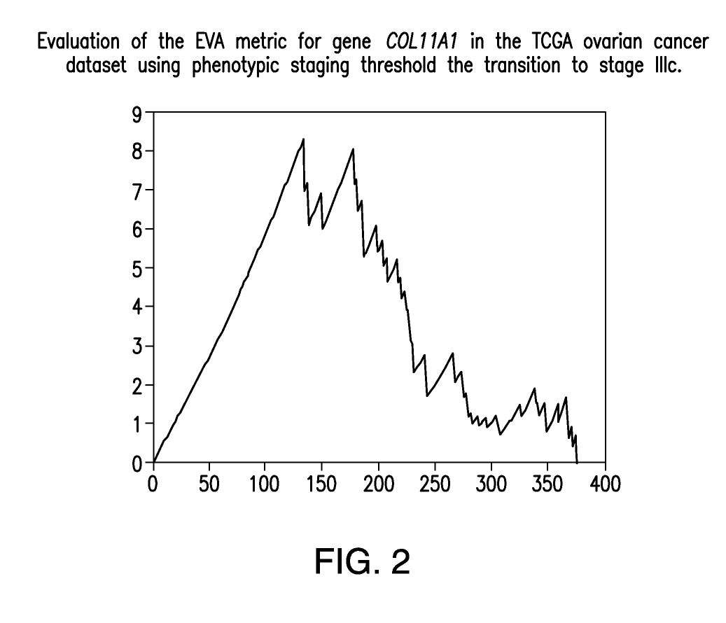

In certain embodiments, the present invention relates to a MAF signature identified by focusing on the cluster of genes associated with the binary (“low stage” versus “high stage”) phenotype (where the particular threshold for low/high staging is dependant on the particular type of cancer) when the genes have their extreme (in most cases, largest) values, but not otherwise, which involved first developing a special measure of association between the gene and the phenotype, which we call “extreme value association” (EVA). Briefly, the EVA metric is the minimum P value of biased partitions over all subsets of samples with highest expression values of the gene. In other words, suppose that there are totally M samples, out of which N are “low stage” and M−N are “high stage,” and we select the m samples with the highest gene expression values. Under the assumption that gene expression values are uncorrelated with the phenotype, the probability that there will be at most n “low stage” samples among the selected m samples is given by the cumulative hypergeometric probability h(x≦n;M,N,m). The EVA metric is then equal to −log₁₀ of the minimum of these probabilities over all possible values of n. For example, assume that there are 250 high-stage samples and 50 low-stage sample for a total of 300 samples. Furthermore, assume that the 100 samples with the highest values of a particular gene contain 99 high-stage samples and one low stage sample. In that case, h(x≦1;300,50,100) can be evaluated using the MATLAB function hypercdf(1,300,50,100)=5×10⁻⁹, resulting in the EVA metric for that gene of at least −log₁₀(5×10⁻⁹)=8.3, e.g. if the 101^{th} sample is also high-stage, then the EVA metric of the gene will be even higher. Note that, once the highest value is reached, the sorting arrangement of the remaining samples is irrelevant, reflecting the hypothesis that only the extreme values are associated with the phenotype. FIG. 2 shows the values of the cumulative hypergeometric probability for the COL11A1 gene using the TCGA ovarian cancer data set and the staging threshold between Mb and IIIc: The maximum (8.31) occurs when m=133. In fact, all 133 samples with the highest COL11A1 expression are at stage IIIc or IV.

We then developed a mechanistic unbiased (only dependent on the phenotype) algorithm, which, when given a gene expression data set for a number of samples labeled “high stage” or “low stage,” leads to a selection of genes that are coordinately overexpressed only in high-stage samples. We first select the top 100 genes that rank highest according to the EVA metric criterion. Using this set of genes only, we perform k-means clustering with gap statistic (Tibshirani R, J R Statist Soc B 63: 411-423). At that step, if indeed the genes are coordinately overexpressed, they will align well in the heat map. This leads to the selection of the samples belonging to the cluster most associated with the high/low stage phenotype—call this the set of “EVA-based samples.” Nearly all samples in that cluster have exceeded the MAF staging threshold, and the very few exceptions could be due to misdiagnosis. Next, we define a “clean” MAF phenotype, contrasting the samples that are: (a) both “EVA based” and “high-stage” against (b) the samples that are both “non EVA-based” and “low stage.” If the number of samples is sufficiently large, this “clean” phenotype provides the sharpest way by which we can identify the genes that are most associated with the observed phenomenon of invasion and/or metastasis-associated coordinated overexpression. We then rank the genes and compute their multiple-test-corrected P values using a heteroscedastic t-test using the “clean” phenotype and select the genes for which P<10⁻³ after Bonferroni correction. Finally, we find the intersection of these selected gene sets over all cancer expression data sets and rank them in terms of fold change.

For a data set with n samples and m probe sets, The EVA algorithm computes n×m cumulative hypergeometric distribution probabilities. This can be quite computationally intensive, so we devised a low-complexity implementation algorithm to dynamically “build” the cumulative hypergeometric distribution for each probe set as the EVA algorithm progresses, as detailed below.

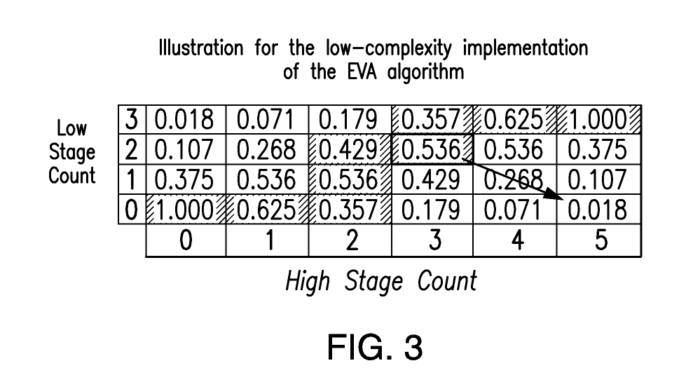

Given a data set with a high-stage samples and b-low stage samples, a (a+1)×(b+1) table of the hypergeometric probabilities corresponding to all possible subsets of the samples is constructed. Then, for each probe set, the samples are sorted according to the expression value of the probe set. This ordering results in a path through the table from the bottom left corner to the top right corner, moving either up or to the right for each sample. At each step in the path, the cumulative probability of encountering the observed number of high stage samples or more is computed by summing the entries diagonally down and to the right of the current cell, including the current cell itself. The algorithm is best demonstrated with a visual example shown in FIG. 3, in which the data set has three low stage samples and five high stage samples in total. Each probe set results in a path through this table, and an example path is displayed here in gray. Letting 1 correspond to a high stage sample and 0 correspond to a low stage sample, this example probe set results in the path 111001011. For the cell in blue, corresponding to the sub-path 111001, the probability of encountering this many high stage samples or more is computed by summing the three probabilities diagonally down and to the right of the blue cell (including itself). In this case, the probability is quite high (82.2%). This cumulative probability is computed for every step along the path, and the minimum of these is the output of the EVA algorithm.

In certain embodiments, the present invention is directed to a biomarker signature that is associated with cancer invasion and/or the presence of MAFs. As used herein, the terms invasion and invasiveness relate to an initial period of metastasis wherein a particular incidence of cancer infiltrates local tissues and dispersion of that cancer begins.

In certain embodiments of the present invention, the biomarker signature of invasion and/or the presence of MAFs includes overexpression of COL11A1.

In certain embodiments, the biomarker signature of invasion and/or the presence of MAFs includes overexpression of COL11A1 and INHBA. In certain embodiments, the biomarker signature of invasion and/or the presence of MAFs includes overexpression of COL11A1 and THBS2. In certain embodiments, the biomarker signature of invasion and/or the presence of MAFs includes overexpression of COL11A1, INHBA, and THBS2.

In certain embodiments, the biomarker signature of invasion and/or the presence of MAFs includes overexpression of at least one of, at least two of, at least three of; at least four of, or at least five, or at least all six of the following proteins: COL11A1 (preferably), COL10A1, COL5A1, COL5A2, COL1A1, and COL1A2.

In certain embodiments, the biomarker signature of invasion and/or the presence of MAFs includes overexpression of at least one of, at least two of, at least three of, at least four of, or at least five, or at least all six of the following proteins: COL11A1 (preferably), COL10A1, COL5A1, COL5A2, COL1A1, and COL1A2; as well as one or more or two or more or three or more of the following: THBS2 (preferably), INHBA (preferably), VCAN, FAP, MMP11, POSTN, ADAM12, LOX, FN1, and SNAI2.

In certain embodiments, the biomarker signature of invasion and/or the presence of MAFs includes overexpression of at least one of, at least two of, at least three of, at least four of, or at least five, or at least all six of the following proteins: COL11A1 (preferably), COL10A1, COL5A1, COL5A2, COL1A1, and COL1A2; as well as one or more or two or more or three or more of the following: THBS2 (preferably), INHBA (preferably), VCAN, FAP, MMP11, POSTN, ADAM12, LOX, FN1, SNAI2; as well as where SNAI1 expression is not significantly altered (e.g., in certain non-limiting embodiments, the SNAI1 gene is methylated). In one specific non-limiting embodiment of the invention, overexpression of COL11A1, THBS2 and INHBA, but not SNAI1, is indicative of invasive progression.

In certain embodiments, the biomarker signature of invasion and/or the presence of MAFs includes overexpression of one, two, or all three of COL11A1, INHBA, and THBS2 in combination with differential expression of one or more miRNAs selected from the group consisting of: hsa-miR-22; hsa-miR-514-1/hsa-miR-514-2|hsa-miR-514-3; hsa-miR-152; hsa-miR-508; hsa-miR-509-1/hsa-miR-509-2/hsa-miR-509-3; hsa-miR-507; hsa-miR-509-1/hsa-miR-509-2; hsa-miR-506; hsa-miR-509-3; hsa-miR-214; hsa-miR-510; hsa-miR-199a-1/hsa-miR199a-2; hsa-miR-21; hsa-miR-513c; and hsa-miR-199b.

In certain embodiments, the biomarker signature of invasion and/or the presence of MAFs includes overexpression of one, two, or all three of COL11A1, INHBA, and THBS2 in combination with differential methylation of one or more genes selected from the group consisting of PRAMS; SNAI1; KRT7; RASSF5; FLJ14816; PPL; CXCR6; SLC12A8; NFATC2; HOM-TES-103; ZNF556; OCIAD2; APS; MGC9712; SLC1A2; HAK; C3orf18; GMPR; and CORO6.

Without being bound by theory, it is believed that the top ranked genes suggest that one feature of the MAF signature is fibroblast activation based on activin signaling. Such signalling is believed to result in some form of altered proteolysis, which eventually leads to an environment rich in collagens COL11A1, COL10A1, COL5A1, COL5A2, COL1A1, and/or COL1A2. Other related genes present in the MAF signature are tissue inhibitor of metalloproteinases-3 (TIMP3), stromelysin-3 (MMP11), and cadherin-11 (CDH11).

Although each of the MAF signature molecules, including miRNAs and methylated genes, such as SNAI1, can serve as a potential therapeutic target, the fact that activin signaling is considered to play a role in the MAF mechanism indicates that follistatin (activin-binding protein) can serve as an invasion and/or metastasis inhibitor, which is exactly what recent research (Talmadge J E, Clin Cancer Res 2008; 14:624-6; Ogino H, Clin Cancer Res 2008; 14:660-7) indicates in the context of individual cancer types. Another approach is to employ mesenchymal-epithelial transition (MET) mediators, such as gene TCF21, which is known to be silenced in several individual types of cancers.

There are several reasons that the MAF signature has not yet been discovered as a multi-cancer invasion and/or metastasis-associated signature, although several other partially overlapping signatures associated with specific cancers have been published. First, each of these other signatures suffer from (a) lack of precise phenotypic definition recognizing that the signature only exists in a subset of tumors that exceed a particular stage. Indeed, if the phenotypic threshold in ovarian cancer were put between stage II and stage III, or between stage III and stage IV, rather than between stage IIIb and stage IIIc, the signature would not be apparent. It is even possible (see below) that wrong selection of the phenotypic threshold would give the reverse result. Second, each cancer type has its own additional features in addition to the MAF signature. For example, in ovarian cancer it is accompanied by sharp downregulation of genes COLEC11, PEG3 and TSPAN8, which is not the case in other cancers. Indeed, one embodiment of the instant invention is the identification of the common multi-cancer “core” signature, from which a universal invasion and/or metastasis-associated biological mechanism can be easier identified. Third and most importantly, the MAF signature is potentially reversible either through a mesenchymal-epithelial transition (MET) or by apoptosis of the MAFs. For example (Ellsworth R E, Clin Exp Metastasis 2009; 26:205-13), in a comparison of metastatic lymph node samples with their corresponding primary breast cancer samples, it was found that COL11A1 had a much higher expression in the primary tumor samples. Such reverse results can hamper data analysis.

The potential reversibility of the MAF signature underscores the fact that the signature is part of a dynamic process and perhaps all invasive and/or metastatic samples have, at some point, been there, but only temporarily, which explains why we only observe it in a subset of them. It has already been recognized that “it is plausible, though hardly proven, that all types of carcinoma cells must undergo a partial or complete EMT to become motile and invasive (Weinberg R A. New York: Garland Science; 2007) p. 600.” This would be particularly exciting, because any invasion and/or metastasis-inhibiting therapeutic intervention targeting the MAF mechanism would be widely applicable to premetastatic tumors across different cancer types, which, until the instant disclosure, has been unrealized goal.

Accordingly, we have shown that, using computational analysis of publicly available biological information, systems biology has revealed the core of a multi-cancer invasion-associated gene expression signature, and the identification of this multi-cancer metastasis associated signature leads to clinical applications, such as invasion and/or metastasis-inhibiting therapeutics. In the near future, a vast amount of additional information will become available, including next generation sequencing, miRNA and methylation information for many cancers, which will allow exciting additional computational research building on this work and clarifying the details of the corresponding complex biological process.

5.2. Assays Employing the MAF Signature

A direct clinical application of the findings described herein concerns the development of high-specificity invasion and/or metastasis-sensing biomarker assay methods. In certain embodiments, such assay methods include, but are not limited: to, nucleic acid amplification assays; nucleic acid hybridization assays; and protein detection assays. In certain embodiments, the assays of the present invention involve combinations of such detection techniques, e.g., but not limited to: assays that employ both amplification and hybridization to detect a change in the expression, such as overexpression or decreased expression, of a gene at the nucleic acid level; immunoassays that detect a change in the expression of a gene at the protein level; as well as combination assays comprising a nucleic acid-based detection step and a protein-based detection step.

“Overexpression”, as used herein, refers to an increase in expression of a gene product relative to a normal or control value, which, in non-limiting embodiments, is an increase of at least about 30% or at least about 40% or at least about 50%, or at least about 100%, or at least about 200%, or at least about 300%, or at least about 400%, or at least about 500%, or at least 1000%.

“Decreased expression”, as used herein, refers to an decrease in expression of a gene product relative to a normal or control value, which, in non-limiting embodiments, is an decrease of at least about 30% or at least about 40% or at least about 50%, at least about 90%, or a decrease to a level where the expression is essentially undetectable using conventional methods.

As used herein, a “gene product” refers to any product of transcription and/or translation of a gene. Accordingly, gene products include, but are not limited to, pre-mRNA, mRNA, and proteins.

In certain embodiments, the present invention provides compositions and methods for the detection of gene expression indicative of all or part of the MAF signature in a sample using nucleic acid hybridization and/or amplification-based assays.

In non-limiting embodiments, the genes/proteins within the MAF signature set forth above constitute at least 10 percent, or at least 20 percent, or at least 30 percent, or at least 40 percent, or at least 50 percent, or at least 60 percent, or at least 70 percent, or at least 80 percent, or at least 90 percent, of the genes/proteins being evaluated in a given assay.

In certain embodiments, the present invention provides compositions and methods for the detection of gene expression indicative of all or part of the MAF signature in a sample using a nucleic acid hybridization assay, wherein nucleic acid from said sample, or amplification products thereof, are hybridized to an array of one or more nucleic acid probe sequences. In certain embodiments, an “array” comprises a support, preferably solid, with one or more nucleic acid probes attached to the support. Preferred arrays typically comprise a plurality of different nucleic acid probes that are coupled to a surface of a substrate in different, known locations. These arrays, also described as “microarrays” or “chips” have been generally described in the art, for example, U.S. Pat. Nos. 5,143,854, 5,445,934, 5,744,305, 5,677,195, 5,800,992, 6,040,193, 5,424,186 and Fodor et al., Science, 251:767-777 (1991).

Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. See U.S. Pat. Nos. 5,770,358, 5,789,162, 5,708,153, 6,040,193 and 5,800,992.

In certain embodiments, the arrays of the present invention can be packaged in such a manner as to allow for diagnostic, prognostic, and/or predictive use or can be an all-inclusive device; e.g., U.S. Pat. Nos. 5,856,174 and 5,922,591.

In certain embodiments, the hybridization assays of the present invention comprise a primer extension step. Methods for extension of primers from solid supports have been disclosed, for example, in U.S. Pat. Nos. 5,547,839 and 6,770,751. In addition, methods for genotyping a sample using primer extension have been disclosed, for example, in U.S. Pat. Nos. 5,888,819 and 5,981,176.

In certain embodiments, the methods for detection of all or a part of the MAF signature in a sample involves a nucleic acid amplification-based assay. In certain embodiments, such assays include, but are not limited to: real-time PCR (for example see Mackay, Clin. Microbial. Infect. 10(3):190-212, 2004), Strand Displacement Amplification (SDA) (for example see Jolley and Nasir, Comb. Chem. High Throughput Screen. 6(3):235-44, 2003), self-sustained sequence replication reaction (3SR) (for example see Mueller et al., Histochem. Cell. Biol. 108(4-5):431-7, 1997), ligase chain reaction (LCR) (for example see Laffler et al., Ann. Biol. Clin. (Paris).51(9):821-6, 1993), transcription mediated amplification (TMA) (for example see Prince et al., J. Viral Hepat. 11(3):236-42, 2004), or nucleic acid sequence based amplification (NASBA) (for example see Romano et al., Clin. Lab. Med. 16(1):89-103, 1996).

In certain embodiments of the present invention, a PCR-based assay, such as, but not limited to, real time PCR is used to detect the presence of a MAF signature in a test sample. In certain embodiments, MAF signature-specific PCR primer sets are used to amplify MAF signature associated RNA and/or DNA targets. Signal for such targets can be generated, for example, with fluorescence-labeled probes. In the absence of such target sequences, the fluorescence emission of the fluorophore can be, in certain embodiments, eliminated by a quenching molecule also operably linked to the probe nucleic acid. However, in the presence of the target sequences, probe binds to template strand during primer extension step and the nuclease activity of the polymerase catalyzing the primer extension step results in the release of the fluorophore and production of a detectable signal as the fluorophore is no longer linked to the quenching molecule. (Reviewed in Bustin, J. Mol. Endocrinol 25, 169-193 (2000)). The choice of fluorophore (e.g., FAM, TET, or Cy5) and corresponding quenching molecule (e.g. BHQ1 or BHQ2) is well within the skill of one in the art and specific labeling kits are commercially available.

In certain embodiments, the present invention provides compositions and methods for the detection of gene expression indicative of all or part of the MAF signature in a sample by detecting changes in concentration of the protein, or proteins, encoded by the genes of interest.

In certain embodiments, the present invention relates to the use of immunoassays to detect modulation of gene expression by detecting changes in the concentration of proteins expressed by a gene of interest. Numerous techniques are known in the art for detecting changes in protein expression via immunoassays. (See The Immunoassay Handbook, 2nd Edition, edited by David Wild, Nature Publishing Group, London 2001.) In certain of such immunoassays, antibody reagents capable of specifically interacting with a protein of interest, e.g., an individual member of the MAF signature, are covalently or non-covalently attached to a solid phase. Linking agents for covalent attachment are known and may be part of the solid phase or derivatized to it prior to coating. Examples of solid phases used in immunoassays are porous and non-porous materials, latex particles, magnetic particles, microparticles, strips, beads, membranes, microtiter wells and plastic tubes. The choice of solid phase material and method of labeling the antibody reagent are determined based upon desired assay format performance characteristics. For some immunoassays, no label is required, however in certain embodiments, the antibody reagent used in an immunoassay is attached to a signal-generating compound or “label”. This signal-generating compound or “label” is in itself detectable or may be reacted with one or more additional compounds to generate a detectable product (see also U.S. Pat. No. 6,395,472 B1). Examples of such signal generating compounds include chromogens, radioisotopes (e.g., ¹²⁵I, ¹³¹I, ³²P, ³H, ³⁵S, and ¹⁴C), fluorescent compounds (e.g., fluorescein and rhodamine), chemiluminescent compounds, particles (visible or fluorescent), nucleic acids, complexing agents, or catalysts such as enzymes (e.g., alkaline phosphatase, acid phosphatase, horseradish peroxidase, beta-galactosidase, and ribonuclease). In the case of enzyme use, addition of chromo-, fluoro-, or lumo-genic substrate results in generation of a detectable signal. Other detection systems such as time-resolved fluorescence, internal-reflection fluorescence, amplification (e.g., polymerase chain reaction) and Raman spectroscopy are also useful in the context of the methods of the present invention.

A “sample” from a subject to be tested according to one of the assay methods described herein may be at least a portion of a tissue, at least a portion of a tumor, a cell, a collection of cells, or a fluid (e.g., blood, cerebrospinal fluid, urine, expressed prostatic fluid, peritoneal fluid, a pleural effusion, peritoneal fluid, etc.). In certain embodiments the sample used in connection with the assays of the instant invention will be obtained via a biopsy. Biopsy may be done by an open or percutaneous technique. Open biopsy is conventionally performed with a scalpel and can involve removal of the entire tumor mass (excisional biopsy) or a part of the tumor mass (incisional biopsy). Percutaneous biopsy, in contrast, is commonly performed with a needle-like instrument either blindly or with the aid of an imaging device, and may be either a fine needle aspiration (FNA) or a core biopsy. In FNA biopsy, individual cells or clusters of cells are obtained for cytologic examination. In core biopsy, a core or fragment of tissue is obtained for histologic examination which may be done via a frozen section or paraffin section.

In certain embodiments of the present invention, the assay methods described herein can be employed to detect the presence of the MAF signature in cancer. In certain embodiments, such cancers can include those involving the presence of solid tumors. In certain embodiments such cancers can include epithelial cancers. In certain embodiments, such cancers can include, for example, but not by way of limitation, cancers of the ovary, stomach, pancreas, duodenum, liver, colon, breast, vagina, cervix, prostate, lung, testicle, oral cavity, esophagus, as well as neuroblastoma and Ewing's sarcoma.

In certain embodiments, the present invention is directed to assay methods allowing for diagnostic, prognostic, and/or predictive use of the MAF signature. For example, but not by way of limitation, the assay methods described herein can be used in a diagnostic context, e.g., where invasive cancer can be diagnosed by detecting all or part of the MAF signature in a sample. In certain non-limiting embodiments, the assay methods described herein can be used in a prognostic context, e.g., where detection of all or part of the MAF signature allows for an assessment of the likelihood of future metastasis, including in those situations where such metastasis is not yet identified. In certain non-limiting embodiments, the assay methods described herein can be used in predictive context, e.g., where detection of all or part of the MAF signature allows for an assessment of the likely benefit of certain types of therapy, such as, but not limited to, neoadjuvant therapy, surgical rescion, and/or chemotherapy.

In certain non-limiting embodiments, the markers and assay methods of the present invention can be used to determine whether a cancer in a subject has progressed to a invasive and/or metastatic form, or has remitted (for example, in response to treatment).

In certain non-limiting embodiments, the markers and assay methods of the present invention can be used to stage a cancer (where clinical staging considers whether invasion has occurred). Such multi-cancer staging is possible due to the fact that the MAF signature is present in a variety of cancers as a marker of invasion which occurs at distinct stages in certain cancers. For example, in certain embodiments, the markers and assay methods of the present invention can be used to stage cancer selected from breast cancer, ovarian cancer, colorectal cancer, and neuroblastoma. In certain embodiments, the markers and assay methods of the present invention can be used to identify when breast carcinoma in situ achieves stage I. In certain embodiments, the markers and assay methods of the present invention can be used to identify when ovarian cancer achieves stage III, and more particularly, stage IIIc. In certain embodiments, the markers and assay methods of the present invention can be used to identify when colorectal cancer achieves stage II. In certain embodiments, the markers and assay methods of the present invention can be used to identify when a neuroblastoma has progressed beyond stage I.

In certain non-limiting embodiments, the markers and assay methods of the present invention can be used to predict drug response in a subject diagnosed with cancer, such as, but not limited to, an epithelial cancer, as at least a portion of the MAF signature has been previously identified as associated with resistance to neoadjuvant chemotherapy in breast cancer (Farmer P, Nat Med 2009; 15:68-74). However, due to the multi-cancer relevance of the MAF signature, which was not appreciated until the filing of the instant disclosure, certain embodiments of the present are directed to using the presence of the MAF signature to predict drug response in a subject diagnosed with an epithelial cancer selected from the group consisting of cancers of the ovary, stomach, pancreas, duodenum, liver, colon, vagina, cervix, prostate, lung, and testicle.

In certain non-limiting embodiments, the MAF signature, or a subset of markers associated with it, can be used to evaluate the contextual (relative) benefit of a therapy in a subject. For example, if a therapeutic decision is based on an assumption that a cancer is localized in a subject, the presence of the MAF signature, or a subset of markers associated with it, would suggest that the cancer is invasive. As a specific, non-limiting embodiment, the relative benefit, to a subject with a malignant tumor, of neoadjuvant chemo- and/or immuno-therapy prior to surgical or radiologic anti-tumor treatment can be assessed by determining the presence of the MAF signature or a subset of markers associated with it, where the presence of the MAF signature or a subset of markers associated with it, is indicative of a decrease in the relative benefit conferred by the neoadjuvant therapy to the subject.

In certain embodiments, the assays of the present invention are capable of detecting coordinated modulation of expression, for example, but not limited to, overexpression, of the genes associated with the MAF signature. In certain embodiments, such detection involves, but is not limited to, detection of the expression of COL11A1, THBS2 and INHBA. In certain embodiments, such detection involves, but is not limited to, detection of the expression of COL11A1 (preferably), COL10A1, COL5A1, COL5A2, COL1A1, and COL1A2; as well as one or more or two or more or three or more of the following: THBS2 (preferably), INHBA (preferably), VCAN, FAP, MMP11, POSTN, ADAM12, LOX, FN1, and SNAI2. For example, but not by way of limitation, a sample from a subject either diagnosed with a cancer or who is being evaluated for the presence or stage of cancer (where the cancer is preferably, but is not limited to, an epithelial cancer) may be tested for the presence of MAF genes and/or overexpression of at least one of, at least two of, at least three of, at least four of, or at least five, or all six of the following proteins: COL11A1 (preferably), COL10A1, COL5A1, COL5A2, COL1A1, and COL1A2; as well as one or more or two or more or three or more of the following: THBS2 (preferably), INHBA (preferably), VCAN, FAP, MMP11, POSTN, ADAM12, LOX, FN1, and SNAI2. Preferably but without limitation SNAI1 expression is not altered (in addition, in certain non-limiting embodiments, the SNAI1 gene is methylated). In one specific non-limiting embodiment of the invention, overexpression of COL11A1, THBS2 and INHBA, but not SNAI1, is indicative of a diagnosis of cancer having invasive and/or metastatic progression.

In certain embodiments, a high-specificity invasion-sensing biomarker assay of the present invention detects overexpression of COL11A1.

In certain embodiments, the high-specificity invasion-sensing biomarker assay detects coordinated overexpression of COL11A1 and INHBA. In certain embodiments the high-specificity invasion-sensing biomarker assay detects coordinated overexpression of COL11A1 and THBS2. In certain embodiments the high-specificity invasion-sensing biomarker assay detects coordinated overexpression of COL11A1, INHBA, and THBS2.

In certain embodiments, the high-specificity invasion-sensing biomarker assay detects coordinated overexpression of one, two, or all three of COL11A1, INHBA, and THBS2 and the expression of one or more of COL10A1, COL5A1, COL5A2, COL1A1, and COL1A2, as well as one or more or two or more or three or more of the following: VCAN, FAP, MMP11, POSTN, ADAM12, LOX, FN1, and SNAI2.

In certain embodiments, the high-specificity invasion-sensing biomarker assay detects coordinated overexpression of one, two, or all three of COL11A1, INHBA, and THBS2 in combination with differential expression of one or more miRNAs selected from the group consisting of: hsa-miR-22; hsa-miR-514-1/hsa-miR-514-2 hsa-miR-514-3; hsa-miR-152; hsa-miR-508; hsa-miR-509-1/hsa-miR-509-2/hsa-miR-509-3; hsa-miR-507; hsa-miR-509-1/hsa-miR-509-2; hsa-miR-506; hsa-miR-509-3; hsa-miR-214; hsa-miR-510; hsa-miR-199a-1/hsa-miR-199a-2; hsa-miR-21; hsa-miR-513c; and hsa-miR-199b.

In certain embodiments, the high-specificity invasion-sensing biomarker assay detects coordinated overexpression of one, two, or all three of COL11A1, INHBA, and THBS2 in combination with differential methylation of one or more genes selected from the group consisting of PRAME; SNAI1; KRT7; RASSF5; FLJ14816; PPL; CXCR6; SLC12A8; NFATC2; HOM-TES-103; ZNF556; OCIAD2; APS; MGC9712; SLC1A2; HAK; C3orf18; GMPR; and CORO6.

Diagnostic kits are also included within the scope of the present invention. More specifically, the present invention includes kits for determining the presence of all or a portion of the MAF signature in a test sample.

Kits directed to determining the presence of all or a portion of the MAF signature in a sample may comprise: a) at least one MAF signature antigen comprising an amino acid sequence selected from the group consisting of) and b) a conjugate comprising an antibody that specifically interacts with said MAF signature antigen attached to a signal-generating compound capable of generating a detectable signal. The kit can also contain a control or calibrator that comprises a reagent which binds to the antigen as well as an instruction sheet describing the manner of utilizing the kit.

In certain embodiments, the kit comprises one or more MAF signature antigen-specific antibody, where the MAF signature antigen comprises or is otherwise derived from a protein encoded by one or more of the following genes: COL11A1 (preferably), COL10A1, COL5A1, COL5A2, COL1A1, and COL1A2, THBS2, INHBA, VCAN, FAP, MMP11, POSTN, ADAM12, LOX, FN1, and SNAI2.

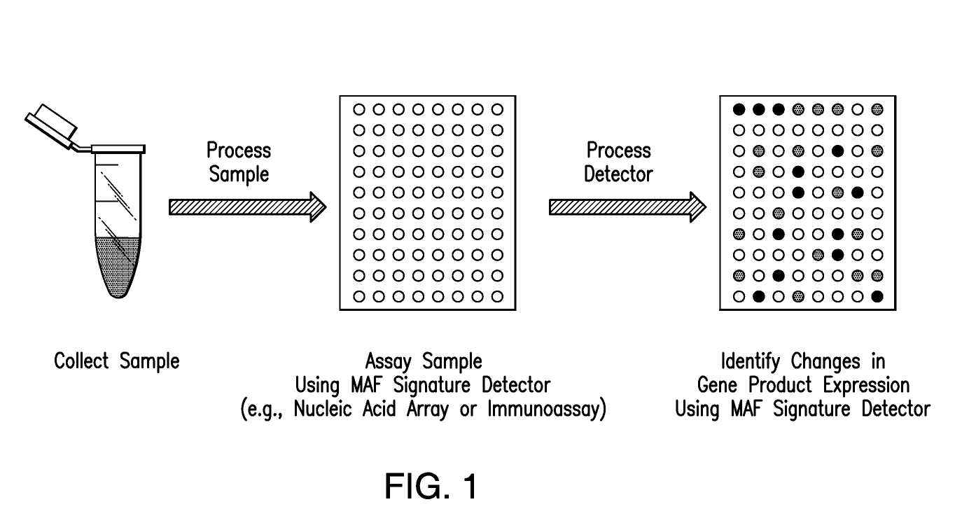

In certain embodiments, the present invention is directed to kits and compositions useful for the detection of MAF signature nucleic acids. In certain embodiments, such kits comprise nucleic acids capable of hybridizing to one or more MAF signature nucleic acids. For example, but not by way of limitation, such kits can be used in connection with hybridization and/or nucleic acid amplification assays to detect MAF signature nucleic acids. FIG. 1 depicts a general strategy that can be used in non-limiting examples of such kits.

In certain embodiments, the hybridization and/or nucleic acid amplification assays that can be employed using the kits of the present invention include, but are not limited to: real-time PCR (for example see Mackay, Clin. Microbiol. Infect. 10(3):190-212, 2004), Strand Displacement Amplification (SDA) (for example see Jolley and Nasir, Comb. Chem. High Throughput Screen. 6(3):235-44, 2003), self-sustained sequence replication reaction (3SR) (for example see Mueller et al., Histochem. Cell. Biol. 108(4-5):431-7, 1997), ligase chain reaction (LCR) (for example see Laffler et al., Ann. Biol. Clin. Paris). 51(9):821-6, 1993), transcription mediated amplification (TMA) (for example see Prince et al., J. Viral Hepat. 11(3):236-42, 2004), or nucleic acid sequence based amplification (NASBA) (for example see Romano et al., Clin. Lab. Med. 16(1):89-103, 1996).

In certain embodiments of the present invention, a kit for detection of MAF signature nucleic acids comprises: (1) a nucleic acid sequence comprising a target-specific sequence that hybridizes specifically to a MAF signature nucleic acid target, and (ii) a detectable label. Such kits can further comprise one or more additional nucleic acid sequence that can function as primers, including nested and/or hemi-nested primers, to mediate amplification of the target sequence. In certain embodiments, the kits of the present invention can further comprise additional nucleic acid sequences function as indicators of amplification, such as labeled probes employed in the context of a real time polymerase chain reaction assay.

The kits of the invention are also useful for detecting multiple MAF signature nucleic acids either simultaneously or sequentially. In such situations, the kit can comprise, for each different nucleic acid target, a different set of primers and one or more distinct labels.

In certain embodiments, the kit comprises nucleic acids (e.g., hybridization probes, primers, or RT-PCR probes) comprising or otherwise derived from one or more of the following genes: COL11A1 (preferably), COL10A1, COL5A1, COL5A2, COL1A1, and COL1A2, THBS2, INHBA, VCAN, FAP, MMP11, POSTN, ADAM12, LOX, FN1, and SNAI2.

Any of the exemplary assay formats described herein and any kit according to the invention can be adapted or optimized for use in automated and semi-automated systems (including those in which there is a solid phase comprising a microparticle), for example as described, e.g., in U.S. Pat. Nos. 5,089,424 and 5,006,309, and in connection with any of the commercially available detection platforms known in the art.

In certain embodiments, the methods, assays, and/or kits of the present invention are directed to the detection of all or a part of the MAP signature wherein such detection can take the form of either a binary, detected/not-detected, result. In certain embodiments, the methods, assays, and/or kits of the present invention are directed to the detection of all or a part of the MAF signature wherein such detection can take the form of a multi-factorial result. For example, but not by way of limitation, such multi-factorial results can take the form of a score based on one, two, three, or more factors. Such factors can include, but are not limited to: (1) detection of a change in expression of a MAF signature gene product, state of methylation, and/or presence of miRNA; (2) the number of MAF signature gene products, states of methylation, and/or presence of miRNAs in a sample exhibiting an altered level; and (3) the extent of such change in MAF signature gene products, states of methylation, and/or presence of miRNAs.

5.3. Methods of Treatment Based on the MAF Signature

In further non-limiting embodiments, the present invention provides for methods of treating a subject, such as, but not limited to, methods comprising performing a diagnostic method as set forth above and then, if a MAF signature is detected in a sample of the subject, recommending that the patient undergo a further diagnostic procedure (e.g. an imaging procedure such as X-ray, ultrasound, computerized axial tomography (CAT scan) or magnetic resonance imaging (MRI)), and/or recommending that the subject be administered therapy with an agent that inhibits invasion and/or metastasis.

In certain non-limiting embodiments of the present invention, a diagnostic method as set forth above is performed and a therapeutic decision is made in light of the results of that assay. For example, but not by way of limitation, a therapeutic decision, such as whether to prescribe neoadjuvant chemo- and/or immuno-therapy prior to surgical or radiologic anti-tumor treatment can be made in light of the results of a diagnostic method as set for the above. The results of the diagnostic method are relevant to the therapeutic decision as the presence of the MAF signature or a subset of markers associated with it, in a sample from a subject indicates a decrease in the relative benefit conferred by the neoadjuvant therapy to the subject since the presence of the MAF signature, or a subset of markers associated with it, is indicative of a cancer that is not localized.

In certain embodiments, a diagnostic method as set forth above is performed and a decision regarding whether to continue a particular therapeutic regimen is made in light of the results of that assay. For example, but not by way of limitation, a decision whether to continue a particular therapeutic regimen, such as whether to continue with a particular chemotherapeutic, radiation therapy, and/or molecular targeted therapy (e.g., a cancer cell-specific antibody therapeutic) can be made in light of the results of a diagnostic method as set for the above. The results of the diagnostic method are relevant to the decision whether to continue a particular therapeutic regimen as the presence of the MAF signature or a subset of markers associated with it, in a sample from a subject can be indicative of the subject's responsiveness to that therapeutic.

5.4. Methods of Drug Discovery Based on the MAF Signature

The instant invention can also be used to develop multi-cancer invasion-inhibiting therapeutics using targets deduced from the biological knowledge provided by the MAF signature. In various non-limiting embodiments, the invention provides for methods of identifying agents that inhibit invasion and/or metastatic dissemination of a cancer in a subject. In certain of such embodiments, the methods comprise exposing a test agent to cancer cells expressing a MAF signature, wherein if the test agent decreases overexpression of genes in the signature, the test agent may be used as a therapeutic agent in inhibiting invasion and/or metastasis of a cancer.

In certain embodiments, the effect of a test agent on the expression of genes in the MAF signature set forth herein may be determined (e.g., but not limited to, overexpression of at least one of, at least two of, at least three of, at least four of, or at least five, or all six of the following proteins: COL11A1 (preferably), COL10A1, COL5A1, COL5A2, COL1A1, and COL1A2; as well as one or more or two or more or three or more of the following: THBS2 (preferably), INHBA (preferably), VCAN, FAP, MMP11, POSTN, ADAM12, LOX, FN1, and SNAI2, and if the test agent decreases overexpression of genes in the signature, the test agent can be used as a therapeutic agent in treating/preventing invasion and/or metastasis of a cancer.

In certain embodiments, the effect of a test agent will be assayed in connection with the expression of COL11A1. In certain embodiments, the effect of a test agent will be assayed in connection with the expression of COL11A1 and INHBA. In certain embodiments, the effect of a test agent will be assayed in connection with the expression of COL11A1 and THBS2. In certain embodiments, the effect of a test agent will be assayed in connection with the expression of COL11A1I, INHBA, and THBS2.

In certain embodiments, the effect of a test agent will be assayed in connection with the expression of one, two, or all three of COL11A1, INHBA, and THBS2 and the expression of one or more of COL10A1, COL5A1, COL5A2, COL1A1, and COL1A2, VCAN, FAP, MMP11, POSTN, ADAM12, LOX, FN1, and SNAI2.

In certain embodiments, the effect of a test agent will be assayed in connection with the expression of one, two, or all three of COL11A1, INHBA, and THBS2 and the expression of one or more miRNAs selected from the group consisting of: hsa-miR-22; hsa-miR-514-1/hsa-miR-514-2|hsa-miR-514-3; hsa-miR-152; hsa-miR-508; hsa-miR-509-1/hsa-miR-509-2/hsa-miR-509-3; hsa-miR-507; hsa-miR-509-1/hsa-miR-509-2; hsa-miR-506; hsa-miR-509-3; hsa-miR-214; hsa-miR-510; hsa-miR-199a-1/hsa-miR199a-2; hsa-miR-21; hsa-miR-513c; and hsa-miR-199b.

In certain embodiments, the effect of a test agent will be assayed in connection with the expression of one, two, or all three of COL11A1, INHBA, and THBS2 and the methylation of one or more genes selected from the group consisting of: PRAME; SNAI1; KRT7; RASSF5; FLJ14816; PPL; CXCR6; SLC12A8; NFATC2; HOM-TES-103; ZNF556; OCIAD2; APS; MGC9712; SLC1A2; HAK; C3orf18; GMPR; and CORO6.

5.5. Detection of Synergistic Gene Pairs

In certain embodiments, as a second step, we identified gene pairs that are most associated with specific members of the MAF signature jointly, but not individually, and therefore they would not appear in the previous investigations. For this task we ranked gene pairs according to their synergy (Anastassiou D, Mol Syst Biol 2007; 3:83) with a MAF signature member, using the computational method in (Watkinson J, Ann NY Acad Sci 2009; 1158:302-13), which could further facilitate biological discovery. We found non-limiting examples of strong validation between the two ovarian cancers, as well as between the two colorectal cancers, but not common to both types of cancer. Of particular interest are the gene pairs (CCL11, MMP2) and (SLAM7, SLAM8), which appear among the top-ranked genes in both colon cancers, and the gene pairs (C7, PDGFRA), (C7, ECM2), (TCF21, ECM2), which appear among the top-ranked genes in both ovarian cancers (TCF21 is a known mesenchymal-epithelial mediator).

In certain embodiments, Mutual Information and Synergy can be evaluated. For example, assuming that two variables, such as the expression levels of two genes G₁ and, G₂ are governed by a joint probability density p₁₂ with corresponding marginals p₁ and p₂ and using simplified notation, the mutual information I(G₁;G₂) is a general measure of correlation and is defined as the expected value

$$E \{ log \frac{{p}_{12}}{{p}_{1} {p}_{2}} \} .$$

The synergy of two variables G₁,G₂ with respect to a third variable G₃ is [14] equal to I(G₁,G₂;G₃)−[I(G₁;G₃)+I(G₂;G₃)], i.e., the part of the association of the pair G₁,G₂ with G₃ that is purely due to a synergistic cooperation between G₁ and G₂ (the “whole” minus the sum of the “parts”).

5.6. Statistical Analysis

In addition to gene expression data, connection between miRNA expression and gene methylation to the MAF signature can also be investigated and employed in the context of the instant invention. For example, but not by way of limitation, P value evaluations for the significance of miRNA expression and gene methylation activity, as well as for synergistic pairs can be performed as follows. We applied a permutation-based approach accounting for multiple test correction: We did 100 permutation experiments of the class labels, saving the corresponding 100 highest values after doing exhaustive search in each permutation experiment. Using the set of these 100 highest-value scores, we obtained the maximum likelihood estimates of the location parameter and the scale parameter of the Gumbel (type-I extreme value) distribution, resulting in a cumulative density function F. The P value of an actual score x₀ is then 1−F(x₀) under the null hypothesis of no association with phenotype. Similarly, for a synergistic pair, we found the top-scoring synergy in 100 data sets that were identical to the original except that the COL11A1 probe values were randomly permuted on each, and the top permuted synergy scores were modelled, as above, with the Gumbel distribution.

### 6. EXAMPLES

### 6.1. Example 1

Since we focus on the cluster of genes associated with the metastasis binary (“low stage” versus “high stage”) phenotype when the genes have their extreme (in most cases, largest) values, but not otherwise, we first developed a special measure of association between the gene and the phenotype, which we call “extreme value association” (EVA). Briefly, the EVA metric is the minimum P value of biased partitions over all subsets of samples with highest expression values of the gene. In other words, suppose that there are totally M samples, out of which N are “low stage” and M−N are “high stage,” and we select the m samples with the highest gene expression values. Under the assumption that gene expression values are uncorrelated with the phenotype, the probability that there will be at most n “low stage” samples among the selected m samples is given by the cumulative hypergeometric probability h(x≦n;M,N,m). The EVA metric is then equal to −log₁₀ of the minimum of these probabilities over all possible values of n. For example, assume that there are 250 high-stage samples and 50 low-stage sample for a total of 300 samples. Furthermore, assume that the 100 samples with the highest values of a particular gene contain 99 high-stage samples and one low stage sample. In that case, h(x≦1;300,50,100) can be evaluated using the MATLAB function hyperedf(1,300,50,100)=5×10⁻⁹, resulting in the EVA metric for that gene of at least −log₁₀(5×10⁻⁹)=8.3, e.g. if the 101^{th} sample is also high-stage, then the EVA metric of the gene will be even higher. Note that, once the highest value is reached, the sorting arrangement of the remaining samples is irrelevant, reflecting the hypothesis that only the extreme values are associated with the phenotype. FIG. 2 shows the values of the cumulative hypergeometric probability for the COL11A1 gene using the TCGA ovarian cancer data set and the staging threshold between IIIb and IIIc: The maximum (8.31) occurs when m=133. In fact, all 133 samples with the highest COL11A1 expression are at stage IIIc or IV.

We then developed a mechanistic unbiased (only dependent on the phenotype) algorithm, which, when given a gene expression data set for a number of samples labeled “high stage” or “low stage,” leads to a selection of genes that are coordinately overexpressed only in high-stage samples. We first select the top 100 genes that rank highest according to the EVA metric criterion. Using this set of genes only, we perform k-means clustering with gap statistic (Tibshirani R, J R Statist Soc B 63: 411-423). At that step, if indeed the genes are coordinately overexpressed, they will align well in the heat map. This leads to the selection of the samples belonging to the cluster most associated with the high/low stage phenotype—call this the set of “EVA-based samples.” Nearly all samples in that cluster have exceeded the MAF staging threshold, and the very few exceptions could be due to misdiagnosis. Next, we define a “clean” MAF phenotype, contrasting the samples that are: (a) both “EVA based” and “high-stage” against (b) the samples that are both “non EVA-based” and “low stage.” If the number of samples is sufficiently large, this “clean” phenotype provides the sharpest way by which we can identify the genes that are most associated with the observed phenomenon of invasion and/or metastasis-associated coordinated overexpression. We then rank the genes and compute their multiple-test-corrected P values using a heteroscedastic t-test using the “clean” phenotype and select the genes for which P<10⁻³ after Bonferroni correction. Finally, we find the intersection of these selected gene sets over all cancer expression data sets and rank them in terms of fold change.

For a data set with n samples and m probe sets, The EVA algorithm computes n×m cumulative hypergeometric distribution probabilities. This can be quite computationally intensive, so we devised a low-complexity implementation algorithm to dynamically “build” the cumulative hypergeometric distribution for each probe set as the EVA algorithm progresses, as detailed below.

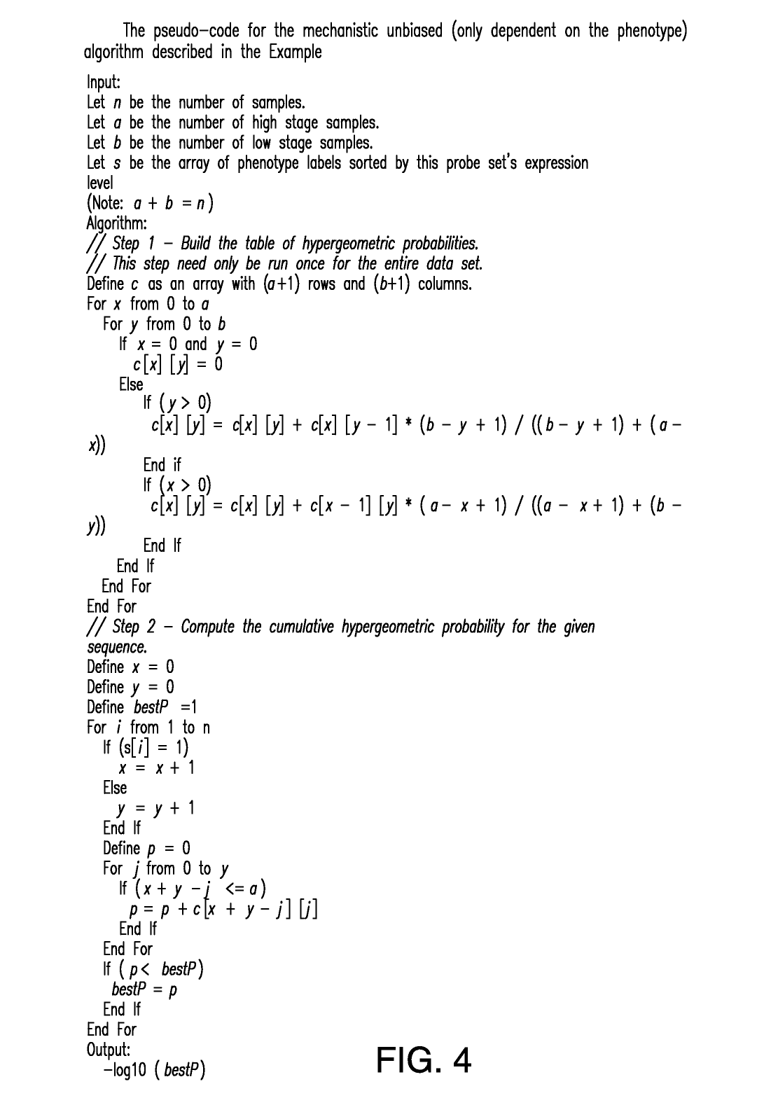

Given a data set with a high-stage samples and b-low stage samples, a (a+1)×(b+1) table of the hypergeometric probabilities corresponding to all possible subsets of the samples is constructed. Then, for each probe set, the samples are sorted according to the expression value of the probe set. This ordering results in a path through the table from the bottom left corner to the top right corner, moving either up or to the right for each sample. At each step in the path, the cumulative probability of encountering the observed number of high stage samples or more is computed by summing the entries diagonally down and to the right of the current cell, including the current cell itself. The algorithm is best demonstrated with a visual example shown in FIG. 3, in which the data set has three low stage samples and five high stage samples in total. Each probe set results in a path through this table, and an example path is displayed here in gray. Letting 1 correspond to a high stage sample and 0 correspond to a low stage sample, this example probe set results in the path 111001011. For the cell in blue, corresponding to the sub-path 111001, the probability of encountering this many high stage samples or more is computed by summing the three probabilities diagonally down and to the right of the blue cell (including itself). In this case, the probability is quite high (82.2%). This cumulative probability is computed for every step along the path, and the minimum of these is the output of the EVA algorithm. The pseudo-code for this algorithm is given in FIG. 4.

We performed the EVA algorithm on four rich gene expression datasets, two from ovarian cancer and two from colorectal cancer (Jorissen R N, Clin Cancer Res 2009; 15:7642-51; Smith J J, Gastroenterology; 138:958-68) for which we had staging information. Using various staging transitions, it became clear that the one that includes samples with the coordinately overexpressed genes is defined as exceeding stage IIIb in ovarian cancer and stage I in colorectal cancer. Interestingly, we realized that the “metastasis-associated genes” identified in (Bignotti E, Am J Obstet Gynecol 2007; 196:245 e1-11) as present in omental metastasis of ovarian cancer were also largely identified in (Tothill R W, Clin Cancer Res 2008; 14:5198-208) as belonging to a “poor prognosis” subtype of ovarian cancer correlated with extensive desmoplasia.

Remarkably, we found that there were multiple genes with P<10⁻¹² common in all four datasets. Table 1 shows a list of these genes with an average log fold change greater than 2. The top ranked gene in terms of fold change was COL11A1 (probe 37892_at), followed by COL10A1, POSTN, ASPN, THBS2, and FAP. Nearly all samples in which these genes were coordinately overexpressed have reached the staging threshold, which is stage II for colon cancer and stage IIIc for ovarian cancer.

TABLE 1
Top-ranked genes associated with high carcinoma stage in ovarian
and colorectal cancers according to the EVA-based algorithm
with Bonferroni corrected P < 10⁻³ in all four data sets

| Probe Set^{a} | Gene | Log FC |
|---|---|---|
| 37892_at | COL11A1 | 3.94 |
| 217428_s_at | COL10A1 | 3.55 |
| 204320_at | COL11A1 | 3.39 |
| 210809_s_at | POSTN | 3.14 |
| 219087_at | ASPN | 2.99 |
| 205941_s_at | COL10A1 | 2.88 |
| 203083_at | THBS2 | 2.81 |
| 209955_s_at | FAP | 2.73 |
| 215446_s_at | LOX | 2.63 |
| 213764_s_at | MFAP5 | 2.61 |
| 210511_s_at | INHBA | 2.52 |
| 215646_s_at | VCAN | 2.5 |
| 209758_s_at | MFAP5 | 2.42 |
| 221730_at | COL5A2 | 2.34 |
| 211571_s_at | VCAN | 2.33 |
| 205713_s_at | COMP | 2.31 |
| 213765_at | MFAP5 | 2.27 |
| 201150_s_at | TIMP3 | 2.25 |
| 221729_at | COL5A2 | 2.24 |
| 212354_at | SULF1 | 2.23 |
| 212489_at | COL5A1 | 2.22 |
| 213790_at | ADAM12 | 2.21 |
| 212488_at | COL5A1 | 2.2 |
| 201147_s_at | TIMP3 | 2.19 |
| 204457_s_at | GAS1 | 2.17 |
| 202952_s_at | ADAM12 | 2.12 |
| 202766_s_at | FBN1 | 2.08 |
| 212344_at | SULF1 | 2.07 |

^{a}Affymetrix probe sets

We then did an extensive literature search aimed at retrospectively identifying other studies where the newly identified signatures could be found within a larger set of genes identified as differentially expressed in various stages of other cancers. We even scrutinized studies in which none of the genes were mentioned in the main text, by looking at their supplementary data and re-ranking particular columns of genes in terms of their fold changes. Although most of the cited references failed to include the newly identified signature even in the context of a larger set of genes, we were able to isolate cancer gene lists from the larger data sets identified in those references with striking similarity to our overall lists. However, it is clear that these references did not appreciate the importance of the newly identified signatures, even if one or more of the genes included in the signatures had previously been included in the context of a larger data set. First, in a breast cancer study (9) comparing ductal carcinomas in situ (DCIS) with invasive ductal carcinoma (IDC), the top-ranked gene was again COL11A1 (probe 37892 at) with fold change of 6.50), while the next highest fold change (4.08) corresponded to another probe of COL11A1, followed by a probe of COL10A1. Second, in a study (Vecchi M, Oncogene 2007; 26:4284-0.94) comparing early gastric cancer (EGC) with advanced gastric cancer (AGC), COL11A1 (probe 37892_at) was again at the top (fold change: 19.2) followed by COL10A1 and FAP. Therefore, in addition to ovarian and colorectal cancers, the MAF signature appears to be present in ductal carcinoma, as well as in gastric cancer. Finally, we realized that COL11A1 has been identified as a potential metastasis-associated gene in other types of cancer as well, such as in lung (Chong I W, Oncol Rep 2006; 16:981-8), and oral cavity (Schmalbach C E, Arch Otolaryngol Head Neck Surg 2004; 130:295-302), suggesting that the MAF signature may be present in a subset of high stage samples of most if not all epithelial cancers. This remarkable consistent strong association of COL11A1 with the phenotype suggests that it could generally be used as a “proxy” of the MAF signature. This, in turn, allowed us to make use of all the publicly available gene expression datasets of cancers of many types, even without any staging information, as long as the MAF signature is present in a sizeable subset of them, aiming at finding the “intersection” of the factors so that we can identify the “core” of the MAF biological mechanism. The data relating to information provided in the corresponding references for breast, gastric and pancreatic cancer is summarized in Table 2.

TABLE 2
Gene lists produced from information provided in the corresponding papers for breast, gastric and pancreatic cancer.

| Breast Cancer, Shuetz et al^{a} | | | Gastric cancer, Vecchi et al^{b} | | | Pancreatic cancer, Badea et al^{c} | | |
|---|---|---|---|---|---|---|---|---|
| Probe Set^{d} | Gene Symbol | Log FC | Probe Set^{d} | Gene Symbol | Log FC | Probe Set^{d} | Gene Symbol | Log FC |
| 37892_at | COL11A1 | 6.50 | 37892_at | COL11A1 | 4.26 | 227140_at | INHBA | 5.15 |
| 204320_at | COL11A1 | 4.08 | 217428_s_at | COL10A1 | 4.15 | 217428_s_at | COL10A1 | 5.00 |
| 217428_s_at | COL10A1 | 4.07 | 209955_s_at | FAP | 3.40 | 1555778_a_at | POSTN | 4.92 |
| 213764_s_at | MFAP5 | 3.73 | 235458_at | HAVCR2 | 3.30 | 212353_at | SULF1 | 4.63 |
| 213909_at | LRRC15 | 3.61 | 204320_at | COL11A1 | 3.28 | 226237_at | COL8A1 | 4.60 |
| 205941_s_at | COL10A1 | 3.52 | 205941_s_at | COL10A1 | 3.21 | 37892_at | COL11A1 | 4.40 |
| 210511_s_at | INHBA | 3.44 | 204052_s_at | SFRP4 | 2.90 | 225681_at | CTHRC1 | 4.38 |
| 202766_s_at | FBN1 | 3.43 | 226930_at | FNDC1 | 2.85 | 202311_s_at | COL1A1 | 4.12 |
| 212353_at | SULF1 | 3.35 | 227140_at | INHBA | 2.77 | 203083_at | THBS2 | 3.97 |
| 218468_s_at | GREM1 | 3.35 | 209875_s_at | SPP1 | 2.77 | 227566_at | HNT | 3.90 |
| 215446_s_at | LOX | 3.22 | 205422_s_at | ITGBL1 | 2.63 | 204619_s_at | CSPG2 | 3.87 |
| 221730_at | COL5A2 | 3.22 | 226311_at | — | 2.63 | 229802_at | WISP1 | 3.80 |
| 218469_at | GREM1 | 3.20 | 222288_at | — | 2.62 | 212464_s_at | FN1 | 3.69 |
| 212489_at | COL5A1 | 3.08 | 231993_at | — | 2.50 | 205713_s_at | COMP | 3.53 |
| 203083_at | THBS2 | 2.99 | 226237_at | COL8A1 | 2.48 | 221729_at | COL5A2 | 3.38 |
| 201505_at | LAMB1 | 2.97 | 223122_s_at | SFRP2 | 2.47 | 209955_s_at | FAP | 3.37 |
| 209955_s_at | FAP | 2.96 | 210511_s_at | INHBA | 2.43 | 229218_at | COL1A2 | 3.16 |
| 209758_s_at | MFAP5 | 2.92 | 203819_s_at | IMP-3 | 2.39 | 209016_s_at | KRT7 | 3.13 |
| 202363_at | SPOCK | 2.91 | 212464_s_at | FN1 | 2.36 | 210004_at | OLR1 | 3.03 |
| 213241_at | NY-REN-58 | 2.90 | 212353_at | SULF1 | 2.35 | 219773_at | NOX4 | 3.02 |
| 205479_s_at | PLAU | 2.89 | 227995_at | — | 2.34 | 218804_at | TMEM16A | 2.90 |
| 206584_at | LY96 | 2.88 | 225681_at | CTHRC1 | 2.30 | 238617_at | — | 2.87 |
| 204475_at | MMP1 | 2.83 | 204457_s_at | GAS1 | 2.27 | 224694_at | ANTXR1 | 2.82 |
| 202952_s_at | ADAM12 | 2.83 | 216442_x_at | FN1 | 2.25 | 228481_at | COX7A1 | 2.77 |
| 201792_at | AEBP1 | 2.81 | 223121_s_at | SFRP2 | 2.23 | 226311_at | ADAMTS2 | 2.76 |
| 204114_at | NID2 | 2.81 | 211719_x_at | FN1 | 2.23 | 201792_at | AEBP1 | 2.68 |
| 213790_at | ADAM12 | 2.80 | 204776_at | THBS4 | 2.18 | 203021_at | SLPI | 2.65 |
| 209156_s_at | COL6A2 | 2.77 | 210495_x_at | FN1 | 2.15 | 227314_at | ITGA2 | 2.58 |
| 219179_at | DACT1 | 2.74 | 202800_at | SLC1A3 | 2.13 | 205499_at | SRPX2 | 2.44 |
| 212488_at | COL5A1 | 2.73 | 214927_at | — | 2.11 | 226997_at | — | 2.41 |
| 219087_at | ASPN | 2.73 | 212354_at | SULF1 | 2.09 | 219179_at | DACT1 | 2.36 |
| 204619_s_at | CSPG2 | 2.70 | 238654_at | LOC147645 | 2.06 | 203570_at | LOXL1 | 2.30 |
| 204337_at | RGS4 | 2.69 | 213943_at | TWIST1 | 2.06 | 201850_at | CAPG | 2.25 |
| 204620_s_at | CSPG2 | 2.69 | 236028_at | IBSP | 2.05 | 222449_at | TMEPAI | 2.19 |
| 212354_at | SULF1 | 2.68 | 228481_at | POSTN | 2.00 | 227276_at | PLXDC2 | 2.16 |

^{a}Breast cancer list indicates genes overexpressed in invasive ductal carcinoma vs. ductal carcinoma in situ.
^{b}Gastric cancer list indicates genes overexpressed in early gastric cancer vs. advanced gastric cancer.
^{c}Pancreatic cancer list indicates genes overexpressed in pancreatic ductal adenocarcinoma vs. normal pancreatic tissue.
^{d}Affymetrix probe sets

As a first step for this task, we identified certain genes, methylation sites, and miRNAs that are consistently highest associated with COL11A1 and the MAF signature. Table 3A shows an aggregate list of genes that are associated with COL11A1, while Tables 3B and 3C relate to methylation sites and miRNA sequences associated with the MAP signature, respectively. The list in Table 3A is very similar to the phenotype-based gene ranking (Table 1). The list of genes in Table 3A that are highly ranked in all datasets, in all cases, were similar to the phenotype-based gene ranking, supporting the hypothesis that COL11A1 can be used as a proxy of the MAF signature. In addition to COL10A1 and a few other collagens, the top ranked genes are thrombospondin-2 (THBS2), inhibin beta A (INHBA), fibroblast activation protein (FAP), leucine rich repeat containing 15 (LRRC15), periostin (POSTN), and a disintegrin and metalloproteinase domain-containing protein 12 (ADAM12). The presence of FAP indicates a general desmoplastic reaction and is not, by itself, sufficient for inferring the MAF signature. Indeed, FAP is occasionally co-expressed with several other EMT-related genes even in healthy tissues. However, COL11A1 was not associated with any of these genes in neither healthy nor low-stage cancerous tissues, further supporting the hypothesis that it can be used as a proxy for the MAF signature. These results indicate that THBS2 and INHBA, top ranked in Table 3A except for collagens, are the most important players in the MAF mechanism.

TABLE 3A
Aggregate list of genes associated with COL11A1
and their corresponding probe set.

| Probe Set | Gene |
|---|---|
| 37892_at | COL11A1 |
| 204320_at | COL11A1 |
| 203083_at | THBS2 |
| 217428_s_at | COL10A1 |
| 205941_s_at | COL10A1 |
| 221729_at | COL5A2 |
| 210511_s_at | INHBA |
| 221730_at | COL5A2 |
| 213909_at | LRRC15 |
| 212488_at | COL5A1 |
| 204619_s_at | VCAN |
| 209955_s_at | FAP |
| 202311_s_at | COL1A1 |
| 221731_x_at | VCAN |
| 203878_s_at | MMP11 |
| 212489_at | COL5A1 |
| 210809_s_at | POSTN |
| 202310_s_at | COL1A1 |
| 204620_s_at | VCAN |
| 202404_s_at | COL1A2 |
| 202952_s_at | ADAM12 |
| 213790_at | ADAM12 |
| 203325_s_at | COL5A1 |
| 215076_s_at | COL3A1 |
| 215446_s_at | LOX |
| 210495_x_at | FN1 |
| 201792_at | AEBP1 |
| 216442_x_at | FN1 |
| 212464_s_at | FN1 |
| 201852_x_at | COL3A1 |
| 212353_at | SULF1 |
| 211719_x_at | FN1 |
| 211161_s_at | COL3A1 |
| 202403_s_at | COL1A2 |
| 202766_s_at | FBN1 |
| 212354_at | SULF1 |
| 219087_at | ASPN |
| 200665_s_at | SPARC |
| 215646_s_at | VCAN |
| 211571_s_at | VCAN |
| 202450_s_at | CTSK |
| 206026_s_at | TNFAIP6 |
| 202765_s_at | FBN1 |
| 203876_s_at | MMP11 |
| 212667_at | SPARC |
| 222020_s_at | HNT |
| 206439_at | EPYC |
| 201069_at | MMP2 |
| 205479_s_at | PLAU |
| 206025_s_at | TNFAIP6 |
| 218469_at | GREM1 |
| 201261_x_at | BGN |
| 213125_at | OLFML2B |
| 201744_s_at | LUM |
| 202998_s_at | ENTPD4 |
| 201438_at | COL6A3 |
| 212344_at | SULF1 |
| 209596_at | MXRA5 |
| 213764_s_at | MFAP5 |
| 204589_at | NUAK1 |
| 217762_s_at | RAB31 |
| 213905_x_at | BGN |
| 201150_s_at | TIMP3 |
| 221541_at | CRISPLD2 |
| 217763_s_at | RAB31 |
| 217430_x_at | COL1A1 |
| 205422_s_at | ITGBL1 |
| 201147_s_at | TIMP3 |
| 218468_s_at | GREM1 |
| 217764_s_at | RAB31 |
| 213765_at | MFAP5 |
| 211668_s_at | PLAU |
| 207173_x_at | CDH11 |
| 213338_at | TMEM158 |
| 209758_s_at | MFAP5 |
| 202363_at | SPOCK1 |
| 201148_s_at | TIMP3 |
| 204051_s_at | SFRP4 |
| 207172_s_at | CDH11 |
| 202283_at | SERPINF1 |
| 209335_at | DCN |
| 204298_s_at | LOX |
| 219655_at | C7orf10 |
| 219561_at | COPZ2 |
| 219773_at | NOX4 |
| 204464_s_at | EDNRA |
| 200974_at | ACTA2 |
| 202273_at | PDGFRB |
| 61734_at | RCN3 |
| 213139_at | SNAI2 |
| 220988_s_at | AMACR |
| 205713_s_at | COMP |
| 201105_at | LGALS1 |
| 213869_x_at | THY1 |
| 202465_at | PCOLCE |
| 208851_s_at | THY1 |
| 209156_s_at | COL6A2 |
| 221447_s_at | GLT8D2 |
| 204114_at | NID2 |
| 205991_s_at | PRRX1 |

TABLE 3B
Aggregate list of methylation sites
associated with the MAF Signature

| Gene | Probe | Hyper/Hypo |
|---|---|---|
| ABCG1 | cg14982472 | Hypo |
| AGR2 | cg21201572 | Hyper |
| AGR2 | cg24426405 | Hyper |
| ALDH3B2 | cg21631409 | Hyper |
| APS | cg05253159 | Hyper |
| ARHGAP9 | cg14338062 | Hypo |
| ARL4 | cg09259772 | Hyper |
| BHMT | cg10660256 | Hypo |
| BRS3 | cg15016628 | Hyper |
| BTBD8 | cg26580095 | Hyper |
| C10orf111 | cg00260778 | Hyper |
| C10orf26 | cg15227982 | Hypo |
| C11orf38 | cg07747336 | Hyper |
| C11orf52 | cg05697249 | Hyper |
| C19orf21 | cg04245402 | Hyper |
| C19orf33 | cg00412772 | Hyper |
| C20orf151 | cg02537838 | Hyper |
| C3orf18 | cg14035045 | Hyper |
| CACHD1 | cg20876010 | Hyper |
| CAV2 | cg11825652 | Hyper |
| CBLC | cg22780475 | Hyper |
| CD3D | cg24841244 | Hypo |
| CFHR5 | cg25840094 | Hyper |
| CFLAR | cg18119407 | Hyper |
| CHRM1 | cg13530039 | Hyper |
| CILP | cg20225681 | Hypo |
| CLDN4 | cg15544036 | Hyper |
| CLUL1 | cg11214889 | Hyper |
| CMTM4 | cg18693704 | Hyper |
| CNKSR1 | cg13553204 | Hyper |
| CORO6 | cg06038133 | Hyper |
| CRISPLD2 | cg07207789 | Hyper |
| CX3CL1 | cg15195412 | Hyper |
| CXCR6 | cg25226014 | Hypo |
| CYP26C1 | cg20322977 | Hypo |
| EDN2 | cg20367961 | Hyper |
| EHF | cg18414381 | Hyper |
| EPHA1 | cg18997129 | Hyper |
| EVI2A | cg23352695 | Hypo |
| EVPL | cg24697031 | Hyper |
| FBXW10 | cg05127924 | Hypo |
| FLJ13841 | cg06022562 | Hyper |
| FLJ14816 | cg17204557 | Hyper |
| FLJ21125 | cg26646411 | Hyper |
| FLJ23235 | cg02131853 | Hyper |
| FLJ31204 | cg12799835 | Hyper |
| FRMD1 | cg00350478 | Hyper |
| FXYD3 | cg02633817 | Hyper |
| FXYD7 | cg22392666 | Hyper |
| GMPR | cg25457331 | Hyper |
| GPR75 | cg14832904 | Hyper |
| GRIK2 | cg26316946 | Hypo |
| GSTP1 | cg05244766 | Hyper |
| HAK | cg15783800 | Hypo |
| HDAC1 | cg24468890 | Hyper |
| HOM-TES-103 | cg00363813 | Hypo |
| HSPB2 | cg12598198 | Hypo |
| IGF1 | cg01305421 | Hypo |
| IL17RE | cg07832674 | Hypo |
| KLB | cg21880903 | Hyper |
| KRT7 | cg09522147 | Hyper |
| LGICZ1 | cg26545162 | Hyper |
| LGP1 | cg08468689 | Hyper |
| LIMD1 | cg04037228 | Hyper |
| LOC126248 | cg26687173 | Hypo |
| LOC284837 | cg01605783 | Hyper |
| MAB21L2 | cg20334738 | Hypo |
| MAGEA5 | cg14107638 | Hyper |
| MEST | cg01888566 | Hyper |
| MEST | cg08077673 | Hyper |
| MEST | cg15164103 | Hyper |
| MFAP2 | cg08477744 | Hypo |
| MGC4618 | cg06154597 | Hyper |
| MGC52423 | cg14036856 | Hyper |
| MGC9712 | cg06194808 | Hyper |
| MGC9712 | cg00411097 | Hyper |
| MPHOSPH9 | cg07732037 | Hypo |
| MYL5 | cg23595927 | Hyper |
| NFATC2 | cg11086066 | Hyper |
| OCIAD2 | cg08942875 | Hyper |
| OSBPL10 | cg15840985 | Hyper |
| PITPNA | cg11719157 | Hyper |
| POF1B | cg24387818 | Hyper |
| PPL | cg12400881 | Hyper |
| PPL | cg16213655 | Hyper |
| PRAME | cg05208878 | Hyper |
| PRELP | cg07947930 | Hyper |
| PROM2 | cg20775254 | Hyper |
| PSMB2 | cg24109894 | Hyper |
| PTPN22 | cg00916635 | Hypo |
| PTPN6 | cg04956511 | Hyper |
| RASSF5 | cg17558126 | Hyper |
| RHOH | cg00804392 | Hypo |
| RPE65 | cg11724759 | Hyper |
| RUNX2 | cg01946401 | Hypo |
| RUNX2 | cg05996042 | Hypo |
| SAMD10 | cg03224418 | Hyper |
| SCGB2A1 | cg16986846 | Hyper |
| SERPINB4 | cg03294557 | Hyper |
| SERPINB5 | cg08411049 | Hyper |
| SF3B14 | cg04809136 | Hyper |
| SFN | cg03421300 | Hyper |
| SH2D3A | cg15055101 | Hyper |
| SHANK2 | cg04396791 | Hypo |
| SLC12A8 | cg14391622 | Hyper |
| SLC1A2 | cg09017174 | Hyper |
| SLC31A2 | cg05706061 | Hyper |
| SLC7A11 | cg06690548 | Hyper |
| SLN | cg17971003 | Hyper |
| SNAI1 | cg26873164 | Hyper |
| SNPH | cg20210637 | Hypo |
| STAP2 | cg05517572 | Hyper |
| SULT1A2 | cg00931491 | Hyper |
| SULT2B1 | cg00698688 | Hyper |
| TCF8 | cg24861272 | Hyper |
| TEAD1 | cg19447966 | Hypo |
| TM4SF5 | cg21066636 | Hyper |
| TNFAIP8 | cg07086380 | Hyper |
| UCN | cg20028470 | Hyper |
| VAMP8 | cg05656364 | Hyper |
| ZCCHC5 | cg03833774 | Hypo |
| ZDHHC11 | cg20584011 | Hyper |
| ZNF511 | cg15856055 | Hyper |
| ZNF556 | cg19636861 | Hyper |

TABLE 3C
Aggregate List of miRNAs associated with the MAF Signature

| Probe | Gene | Up_Down |
|---|---|---|
| A_25_P00010204 | hsa-miR-22 | Up |
| A_25_P00012685 | hsa-miR-514-1\|hsa-miR-514-2\|hsa-miR-514-3 | Down |
| A_25_P00012196 | hsa-miR-152 | Up |
| A_25_P00013178 | hsa-miR-22 | Up |
| A_25_P00011039 | hsa-miR-508 | Down |
| A_25_P00012678 | hsa-miR-509-1\|hsa-miR-509-2\|hsa-miR-509-3 | Down |
| A_25_P00010205 | hsa-miR-22 | Up |
| A_25_P00011112 | hsa-miR-507 | Down |
| A_25_P00011111 | hsa-miR-507 | Down |
| A_25_P00014175 | hsa-miR-509-1\|hsa-miR-509-2 | Down |
| A_25_P00011037 | hsa-miR-506 | Down |
| A_25_P00012684 | hsa-miR-514-1\|hsa-miR-514-2\|hsa-miR-514-3 | Down |
| A_25_P00014918 | hsa-miR-509-3 | Down |
| A_25_P00012677 | hsa-miR-509-1\|hsa-miR-509-2\|hsa-miR-509-3 | Down |
| A_25_P00013059 | hsa-miR-509-3 | Down |
| A_25_P00012106 | hsa-miR-214 | Up |
| A_25_P00011038 | hsa-miR-506 | Down |
| A_25_P00012107 | hsa-miR-214 | Up |
| A_25_P00012682 | hsa-miR-510 | Down |
| A_25_P00010700 | hsa-miR-199a-1\|hsa-miR-199a-2 | Up |
| A_25_P00012674 | hsa-miR-509-1\|hsa-miR-509-2 | Down |
| A_25_P00012195 | hsa-miR-152 | Up |
| A_25_P00010976 | hsa-miR-21 | Up |
| A_25_P00014974 | hsa-miR-513c | Down |
| A_25_P00010699 | hsa-miR-199b | Up |
| A_25_P00014557 | hsa-miR-214 | Up |
| A_25_P00012681 | hsa-miR-510 | Down |
| A_25_P00011040 | hsa-miR-508 | Down |
| A_25_P00010698 | hsa-miR-199b | Up |
| A_25_P00014970 | hsa-miR-513b | Down |
| A_25_P00010701 | hsa-miR-199a-1\|hsa-miR-199a-2 | Up |
| A_25_P00014973 | hsa-miR-513c | Down |
| A_25_P00010407 | hsa-miR-409 | Up |
| A_25_P00013174 | hsa-miR-21 | Up |
| A_25_P00013335 | hsa-miR-214 | Up |
| A_25_P00013173 | hsa-miR-21 | Up |
| A_25_P00013177 | hsa-miR-22 | Up |
| A_25_P00010408 | hsa-miR-409 | Up |
| A_25_P00013065 | hsa-miR-934 | Up |
| A_25_P00010585 | hsa-miR-382 | Up |
| A_25_P00012666 | hsa-miR-508 | Down |
| A_25_P00010589 | hsa-miR-132 | Up |
| A_25_P00014822 | hsa-miR-31 | Up |
| A_25_P00012019 | hsa-miR-31 | Up |
| A_25_P00014828 | hsa-miR-199a-1\|hsa-miR-199a-2\|hsa-miR-199b | Up |
| A_25_P00010885 | hsa-miR-181a-1 | Up |
| A_25_P00010588 | hsa-miR-132 | Up |
| A_25_P00010382 | hsa-miR-127 | Up |
| A_25_P00010381 | hsa-miR-127 | Up |
| A_25_P00012320 | hsa-miR-370 | Up |
| A_25_P00014844 | hsa-miR-142 | Up |
| A_25_P00012181 | hsa-miR-142 | Up |
| A_25_P00014887 | hsa-miR-513a-1\|hsa-miR-513a-2 | Down |
| A_25_P00012665 | hsa-miR-508 | Down |
| A_25_P00013215 | hsa-miR-31 | Up |
| A_25_P00014972 | hsa-miR-513c | Down |
| A_25_P00012337 | hsa-miR-379 | Up |
| A_25_P00012338 | hsa-miR-379 | Up |
| A_25_P00014969 | hsa-miR-513b | Down |
| A_25_P00011016 | hsa-miR-142 | Up |
| A_25_P00014846 | hsa-miR-150 | Up |
| A_25_P00012451 | hsa-miR-452 | Up |
| A_25_P00013171 | hsa-miR-20a | Down |
| A_25_P00014968 | hsa-miR-513b | Down |
| A_25_P00010992 | hsa-miR-645 | Up |
| A_25_P00010490 | hsa-miR-150 | Up |
| A_25_P00014847 | hsa-miR-150 | Up |
| A_25_P00014215 | hsa-miR-551b | Up |
| A_25_P00013214 | hsa-miR-31 | Up |
| A_25_P00014853 | hsa-miR-381 | Up |
| A_25_P00014891 | hsa-miR-513a-1\|hsa-miR-513a-2 | Down |
| A_25_P00012082 | hsa-miR-10b | Down |
| A_25_P00010343 | hsa-miR-219-1\|hsa-miR-219-2 | Down |
| A_25_P00014894 | hsa-miR-551b | Up |
| A_25_P00012357 | hsa-miR-342 | Up |
| A_25_P00012316 | hsa-miR-376c | Up |
| A_25_P00013937 | hsa-miR-142 | Up |
| A_25_P00010975 | hsa-miR-21 | Up |
| A_25_P00010342 | hsa-miR-219-1\|hsa-miR-219-2 | Down |
| A_25_P00014829 | hsa-miR-199a-1\|hsa-miR-199a-2\|hsa-miR-199b | Up |
| A_25_P00014971 | hsa-miR-513c | Down |
| A_25_P00012317 | hsa-miR-376c | Up |
| A_25_P00010761 | hsa-miR-27b | Up |
| A_25_P00010882 | hsa-miR-23b | Up |
| A_25_P00012200 | hsa-miR-153-1\|hsa-miR-153-2 | Down |
| A_25_P00010182 | hsa-miR-381 | Up |
| A_25_P00012270 | hsa-miR-155 | Up |
| A_25_P00010275 | hsa-miR-376a-1\|hsa-miR-376a-2 | Up |
| A_25_P00010583 | hsa-miR-154 | Up |
| A_25_P00010677 | hsa-miR-24-1\|hsa-miR-24-2 | Up |
| A_25_P00012193 | hsa-miR-145 | Up |
| A_25_P00012192 | hsa-miR-145 | Up |
| A_25_P00012134 | hsa-miR-224 | Up |
| A_25_P00010125 | hsa-miR-377 | Up |
| A_25_P00014886 | hsa-miR-513a-1\|hsa-miR-513a-2 | Down |
| A_25_P00011018 | hsa-miR-136 | Up |
| A_25_P00010276 | hsa-miR-376a-1\|hsa-miR-376a-2 | Up |
| A_25_P00013170 | hsa-miR-20a | Down |
| A_25_P00010755 | hsa-miR-34c | Down |
| A_25_P00010963 | hsa-miR-133b | Up |
| A_25_P00010775 | hsa-miR-449b | Down |
| A_25_P00010993 | hsa-miR-645 | Up |
| A_25_P00010676 | hsa-miR-24-1\|hsa-miR-24-2 | Up |
| A_25_P00010220 | hsa-miR-449a | Down |
| A_25_P00012133 | hsa-miR-224 | Up |
| A_25_P00012083 | hsa-miR-10b | Down |
| A_25_P00010078 | hsa-miR-146a | Up |
| A_25_P00012472 | hsa-miR-488 | Down |
| A_25_P00010994 | hsa-miR-645 | Up |
| A_25_P00012362 | hsa-miR-337 | Up |
| A_25_P00010465 | hsa-miR-34b | Down |
| A_25_P00010756 | hsa-miR-34c | Down |
| A_25_P00011002 | hsa-miR-9-1\|hsa-miR-9-2\|hsa-miR-9-3 | Down |
| A_25_P00010221 | hsa-miR-449a | Down |
| A_25_P00010604 | hsa-miR-411 | Up |
| A_25_P00014837 | hsa-miR-27b | Up |
| A_25_P00012358 | hsa-miR-342 | Up |
| A_25_P00010206 | hsa-miR-592 | Down |
| A_25_P00014053 | hsa-miR-452 | Up |
| A_25_P00012271 | hsa-miR-155 | Up |
| A_25_P00014832 | hsa-miR-181a-2\|hsa-miR-181a-1 | Down |
| A_25_P00011017 | hsa-miR-136 | Up |
| A_25_P00010126 | hsa-miR-377 | Up |
| A_25_P00011083 | hsa-miR-431 | Up |
| A_25_P00010605 | hsa-miR-411 | Up |
| A_25_P00010837 | hsa-miR-30e | Down |
| A_25_P00012312 | hsa-miR-362 | Down |
| A_25_P00010103 | hsa-miR-299 | Up |
| A_25_P00013295 | hsa-miR-7-1 | Down |
| A_25_P00010316 | hsa-miR-9-1\|hsa-miR-9-2\|hsa-miR-9-3 | Down |
| A_25_P00012319 | hsa-miR-370 | Up |
| A_25_P00010071 | hsa-let-7b | Up |
| A_25_P00011381 | hsa-miR-641 | Down |
| A_25_P00012097 | hsa-miR-183 | Down |
| A_25_P00012021 | hsa-miR-32 | Down |
| A_25_P00012361 | hsa-miR-337 | Up |
| A_25_P00010613 | hsa-miR-20a | Down |
| A_25_P00010315 | hsa-miR-9-1\|hsa-miR-9-2\|hsa-miR-9-3 | Down |
| A_25_P00013163 | hsa-miR-19b-1 | Down |
| A_25_P00010070 | hsa-let-7b | Up |
| A_25_P00010648 | hsa-miR-551b | Up |
| A_25_P00010464 | hsa-miR-34b | Down |
| A_25_P00012001 | hsa-miR-26b | Down |
| A_25_P00010776 | hsa-miR-449b | Down |
| A_25_P00012412 | hsa-miR-196b | Up |

### 6.2. Example 2

As a second step, we identified gene pairs that are most associated with COL11A1 jointly, but not individually, and therefore they would not appear in the previous list. For this task we ranked gene pairs according to their synergy (Anastassiou D, Mol Syst Biol 2007; 3:83) with COL11A1, using the computational method in (Watkinson J, Ann NY Acad Sci 2009; 1158:302-13), which could further facilitate biological discovery. We found strong validation between the two ovarian cancers, as well as between the two colorectal cancers, but not common to both types of cancer. Of particular interest are the gene pairs (CCL11, MMP2) and (SLAM7, SLAMS), which appear among the top-ranked genes in both colon cancers, and the gene pairs (C7, PDGFRA), (C7, ECM2), (TCF21, ECM2), which appear among the top-ranked genes in both ovarian cancers (TCF21 is a known mesenchymal-epithelial mediator).

Mutual Information and Synergy was evaluated as follows. Assuming that two variables, such as the expression levels of two genes G₁ and, G₂ are governed by a joint probability density p₁₂ with corresponding marginals p₁ and p₂ and using simplified notation, the mutual information I(G₁;G₂) is a general measure of correlation and is defined as the expected value

$$E \{ log \frac{{p}_{12}}{{p}_{1} {p}_{2}} \} .$$

The synergy of two variables G₁,G₂ with respect to a third variable G₃ is [14] equal to I(G₁,G₂;G₃)−[I(G₁;G₃)+I(G₂;G₃)], i.e., the part of the association of the pair G₁,G₂ with G₃ that is purely due to a synergistic cooperation between G₁ and G₂ (the “whole” minus the sum of the “parts”).

### 6.2. Example 3

In addition to gene expression data, connection between miRNA expression and gene methylation to the MAF signature were also investigated. P value evaluations for the significance of miRNA expression and gene methylation activity, as well as for synergistic pairs were performed as follows. We applied a permutation-based approach accounting for multiple test correction: We did 100 permutation experiments of the class labels, saving the corresponding 100 highest values after doing exhaustive search in each permutation experiment. Using the set of these 100 highest-value scores, we obtained the maximum likelihood estimates of the location parameter and the scale parameter of the Gumbel (type-I extreme value) distribution, resulting in a cumulative density function F. The P value of an actual score x₀ is then 1−F(x₀) under the null hypothesis of no association with phenotype. Similarly, for the synergistic pair, we found the top-scoring synergy in 100 data sets that were identical to the original except that the COL11A1 probe values were randomly permuted on each, and the top permuted synergy scores were modelled, as above, with the Gumbel distribution.

We only had miRNA and methylation data available for the TCGA ovarian data set. Using as measure the mutual information with COL11A1, we found many statistically significant miRNAs, among them hsa-miR-22 and hsa-miR-152, as well as differentially methylated genes, such as SNAI1 and PRAME, suggesting a particularly complex biological mechanism (correlation with the MAF phenotype led to essentially the same lists with lower significance). Table 4 contains a list of the miRNAs, while Table 5 contains a list of the methylated genes (multiple test corrected P<10⁻¹⁶ in both cases, see above). SNAI1 (snail) methylation is particularly important as the gene is known as one of the most important EMT-related transcription factors. Instead, the strongest MAF-associated transcription factor is AEBP1, making it a particularly interesting potential target. Many of the other EMT-related transcription factors, such as SNAI2, TWIST1, and ZEB1 are often overexpressed in the MAF signature, but SNAI1 is not (and, at least in ovarian carcinoma in which we have methylation data, this is due to its differentially methylated status). Thus, the lack of SNAI1 expression is an important distinguishing feature of the MAF signature in certain embodiments, in which we observed neither SNAI1 overexpression nor CDH1 (E-cadherin) downregulation.

TABLE 4
Top ranked (multiple-test corrected P < 10⁻¹⁶) differentially
expressed miRNAs in MAF signature in the TCGA ovarian cancer
data set in terms of their association with COL11A1.

| | | Up/Down |
|---|---|---|
| miRNA | MI | Regulated |
| hsa-miR-22 | 0.204 | Up |
| hsa-miR-514-1\|hsa-miR-514-2\|hsa-miR-514-3 | 0.193 | Down |
| hsa-miR-152 | 0.187 | Up |
| hsa-miR-508 | 0.168 | Down |
| hsa-miR-509-1\|hsa-miR-509-2\|hsa-miR-509-3 | 0.164 | Down |
| hsa-miR-507 | 0.152 | Down |
| hsa-miR-509-1\|hsa-miR-509-2 | 0.147 | Down |
| hsa-miR-506 | 0.146 | Down |
| hsa-miR-509-3 | 0.144 | Down |
| hsa-miR-214 | 0.128 | Up |
| hsa-miR-510 | 0.116 | Down |
| hsa-miR-199a-1\|hsa-miR-199a-2 | 0.115 | Up |
| hsa-miR-21 | 0.112 | Up |
| hsa-miR-513c | 0.108 | Down |
| hsa-miR-199b | 0.103 | Up |

TABLE 5
Top ranked (multiple-test corrected P < 10⁻¹⁶) differentially
methylated genes in MAF signature in the TCGA ovarian cancer
data set in terms of their association with COL11A1.

| Methylation site | MI | Hyper-/Hypomethylated |
|---|---|---|
| PRAME | 0.223 | Hyper |
| SNAI1 | 0.183 | Hyper |
| KRT7 | 0.158 | Hyper |
| RASSF5 | 0.157 | Hyper |
| FLJ14816 | 0.155 | Hyper |
| PPL | 0.155 | Hyper |
| CXCR6 | 0.153 | Hypo |
| SLC12A8 | 0.148 | Hyper |
| NFATC2 | 0.148 | Hyper |
| HOM-TES-103 | 0.147 | Hypo |
| ZNF556 | 0.147 | Hyper |
| OCIAD2 | 0.146 | Hyper |
| APS | 0.142 | Hyper |
| MGC9712 | 0.139 | Hyper |
| SLC1A2 | 0.136 | Hyper |
| HAK | 0.131 | Hypo |
| C3orf18 | 0.130 | Hyper |
| GMPR | 0.130 | Hyper |
| CORO6 | 0.128 | Hyper |

Various references are cited herein which are hereby incorporated by reference in their entireties.

What is claimed is:

1. A method of diagnosing invasive cancer in a subject comprising determining, in a sample from the subject, the expression level, relative to a normal subject, of a COL11A1 gene product wherein overexpression of a COL11A1 gene product indicates that the subject has invasive cancer

2. The method of claim 1 wherein the expression level, relative to a normal subject, of one or more of COL5A2, VCAN, SPARC, THBS2, FBN1, COL1A2, COL5A1, FAP, AEBP1, and CTSK is determined and wherein the overexpression of a COL11A1 gene product and of one or more of a COL5A2, VCAN, SPARC, THBS2, FBN1, COL1A2, COL5A1, FAP, AEBP1, and CTSK gene product indicate that a subject has invasive cancer.

3. The method of claim 1 where the expression level is determined by a method comprising processing the sample so that cells in the sample are lysed.

4. The method of claim 3, comprising the further step of at least partially purifying cell gene products and exposing said proteins to a detection agent.

5. The method of claim 3, comprising the further step of at least partially purifying cell nucleic acid and exposing said nucleic acid to a detection agent.

6. The method of claim 1, comprising the further step of determining the expression level of SNAI1, where a determination that SNAI1 is not overexpressed and the other gene products are overexpressed indicates that the subject has invasive cancer.

7. A method of developing a prognosis relating to a cancer in a subject comprising determining, in a sample from the subject, the expression level, relative to a normal subject, of at least one gene product selected from the group consisting of COL11A1, COL10A1, COL5A1, COL5A2, COL1A1, and COL1A2, and at least one gene product selected from the group consisting of THBS2, INHBA, VCAN, FAP, MMP11, POSTN, ADAM12, LOX, FN1, SPARC, FBN1, AEBP1, CTSK, and SNAI2, wherein overexpression of said gene products indicates a likelihood that the cancer present in the subject will become metastatic.

8. The method of claim 7 where the expression level is determined by a method comprising processing the sample so that cells in the sample are lysed.

9. The method of claim 8, comprising the further step of at least partially purifying cell gene products and exposing said proteins to a detection agent.

10. The method of claim 8, comprising the further step of at least partially purifying cell nucleic acid and exposing said nucleic acid to a detection agent.

11. The method of claim 7, comprising the further step of determining the expression level of SNAI1, where a determination that SNAI1 is not overexpressed and the other gene products are overexpressed indicates a likelihood that the cancer present in the subject will become metastatic.

12. A method of treating a subject, comprising performing the diagnostic method of claim 1, and, where the protein is overexpressed, recommending that the patient not undergo neoadjuvant treatment.

13. A method of identifying an agent that inhibits cancer invasion in a subject, comprising exposing a test agent to cancer cells expressing a metastasis associated fibroblast signature, wherein if the test agent decreases overexpression of genes in the signature, the test agent may be used as a therapeutic agent in inhibiting invasion of a cancer.

14. The method of claim 13, wherein the metastasis associated fibroblast signature comprises overexpression of at least one gene product selected from the group consisting of COL11A1, COL10A1, COL5A1, COL5A2, COL1A1, and COL1A2, and at least one gene product selected from the group consisting of THBS2, INHBA, VCAN, FAP, MMP11, POSTN, ADAM12, LOX, FN1, SPARC, FBN1, AEBP1, CTSK, and SNAI2.

15. A kit comprising:
(a) a labeled reporter molecule capable of specifically interacting with a metastasis associated fibroblast signature gene product;
(b) a control or calibrator reagent, and
(c) instructions describing the manner of utilizing the kit.

16. The kit of claim 15 comprising:
(a) a conjugate comprising an antibody that specifically interacts with a metastasis associated fibroblast signature antigen attached to a signal-generating compound capable of generating a detectable signal;
(b) a control or calibrator reagent, and
(c) instructions describing the manner of utilizing the kit.

17. The kit of claim 16 comprising a metastasis associated fibroblast signature antigen-specific antibody, where the metastasis associated fibroblast signature antigen bound by said antibody comprises or is otherwise derived from a protein encoded by one or more of the following genes: COL11A1, COL10A1, COL5A1, COL5A2, COL1A1, COL1A2, THBS2, INHBA, VCAN, FAP, MMP11, POSTN, ADAM12, LOX, FN1, and SNAI2

18. The kit of claim 15 comprising:
(a) a nucleic acid capable of hybridizing to a metastasis associated fibroblast signature nucleic acid;
(b) a control or calibrator reagent; and
(c) instructions describing the manner of utilizing the kit.

19. The kit of claim 15 comprising:
(a) a nucleic acid sequence comprising
(i) a target-specific sequence that hybridizes specifically to a metastasis associated fibroblast signature nucleic acid, and
(ii) a detectable label;
(b) a primer nucleic acid sequence;
(c) a nucleic acid indicator of amplification; and.
(d) instructions describing the manner of utilizing the kit.

20. The kit of claim 19 wherein the nucleic acid that hybridizes specifically to a metastasis associated fibroblast signature nucleic acid comprising or otherwise derived from one of the following genes: COL11A1, COL10A1, COL5A1, COL5A2, COL1A1, COL1A2, THBS2, INHBA, VCAN, FAP, MMP11, POSTN, ADAM12, LOX, FN1, SPARC, FBN1, AEBP1, CTSK, and SNAI2.
